(19) United States
(12) Patent Application Publication
(10) Pub. No.: US 2024/0269161 A1
(43) Pub. Date: Aug. 15, 2024

(54) METHODS AND COMPOSITIONS FOR TAU REDUCTION GENE THERAPY

(71) Applicant: THE BOARD OF REGENTS OF THE UNIVERSITY OF TEXAS SYSTEM, Austin, TX (US)

(72) Inventor: Rachel M. BAILEY, Austin, TX (US)

(73) Assignee: THE BOARD OF REGENTS OF THE UNIVERSITY OF TEXAS SYSTEM, Austin, TX (US)

(21) Appl. No.: 18/569,019

(22) PCT Filed: Jun. 27, 2022
(86) PCT No.: PCT/US2022/035091

§ 371 Date: Dec. 11, 2023

Related U.S. Application Data
(60) Provisional application No. 63/215,833, filed on Jun. 28, 2021.
(60) Provisional application No. 63/267,440, filed on Feb. 2, 2022.
(60) Provisional application No. 63/342,240, filed on May 16, 2022.

(51) Int. Cl. A61K 31/713; A61P 25/28; C12N 15/113; C12N 15/86

(52) U.S. Cl. CPC A61K 31/713; A61P 25/28; C12N 15/113; C12N 15/86; C12N 2310/141; C12N 2750/14143

(57) ABSTRACT

The present disclosure provides methods and compositions for the treatment of tauopathies, such as, for example, Alzheimer's Disease or FTDP-17. The methods and compositions of the present disclosure comprise isolated nucleic acid molecules, rAAV vectors and rAAV viral vectors comprising polynucleotide sequences encoding for artificial micro RNAs (amiRNAs) directed against MAPT.

### CROSS REFERENCE TO RELATED APPLICATIONS

This application claims priority to and the benefit of U.S. Provisional Patent Applications No. 63/215,833, filed on Jun. 28, 2021, No. 63/267,440, filed Feb. 2, 2022, and No. 63/342,240, filed May 16, 2022, the contents of each of which are incorporated herein by reference in their entireties.

### INCORPORATION BY REFERENCE OF SEQUENCE LISTING

The instant application contains a Sequence Listing which has been submitted in ASCII format via EFS-Web and is hereby incorporated by reference in its entirety. Said ASCII copy, created on Jun. 6, 2022, is named “TAYS-013_001WO_Seq_Listing_ST25.txt” and is 148,953 bytes in size.

### FIELD

The present disclosure provides methods and compositions for the treatment of Tauopathies, including Alzheimer's Disease (AD) and Frontotemporal Dementia and Parkinsonism Linked To Chromosome 17 (FTDP-17).

The methods and compositions of the present disclosure comprise isolated nucleic acid molecules, recombinant adeno-associated virus (rAAV) vectors and rAAV viral vectors comprising polynucleotide sequences encoding for artificial micro RNAs (amiRNAs) directed against MAPT.

### BACKGROUND

A common pathological hallmark of neurodegenerative disease is the misfolding and deposition of specific proteins into insoluble proteinaceous deposits in the CNS accompanied by a progressive loss of neurons in the affected regions. Tauopathies are a group of neurodegenerative disorders that are broadly defined by the aggregation of hyperphosphorylated, filamentous tau protein. The most common tauopathy is Alzheimer's Disease (AD). While AD is characterized by the aggregation of both b-amyloid as plaques and hyperphosphorylated tau as neurofibrillary tangles (NFTs), only NFT pathology closely correlates with cognitive decline. Tau fibrils stably propagate and spread tau pathology transcellularly from different brain regions, depending on the disease (Braak et al. Acta Neuropathol 82, 239-259, doi: 10.1007/bf00308809 (1991); Braak et al., Acta Neuropathol121, 589-595, doi: 10.1007/s00401-011-0825-z (2011); Irwin et al., Parkinsonism Re at Disord 22 Suppl 1, S29-33, doi: 10.1016/j.parkreldis.2015.09.020S1353-8020(15)00395-8 [pii] (2016)).

Though the MAPT gene encoding tau is not genetically linked to AD, mutations in MAPT cause other tauopathies such as Frontotemporal Dementia and Parkinsonism Linked To Chromosome 17 (FTDP-17) (Hutton et al. Nature 393, 702-705, doi: 10.1038/31508 (1998): Spillantini et al. Proc Natl Acad Sci USA 95, 7737-7741, doi: 10.1073/pnas.95.13.7737 (1998)) showing that disrupting tau homeostasis is sufficient to cause neurodegeneration.

Over the last decade there have been significant advances towards molecular therapies to target proteins that misfold for degradation. Most approaches exploit the endogenous RNAi mechanism by using one of two major RNAi platforms have been under development: oligonucleotides and gene therapy. The former has moved faster into the clinic with antisense oligonucleotides (ASOs) that target huntingtin to treat Huntington's Disease (HD) (Kordasiewicz et al. Neuron 74, 1031-1044, doi: 10.1016/j.neuron.2012.05.009 (2012): NCT03761849), SODI to treat Amyotrophic Lateral Sclerosis (ALS) (Miller et al. Lancet Neural 12, 435-442, doi: 10.1016/S1474-4422(13)70061-9 (2013); NCT02623699), C90rf72 to treat ALS (Mccampbell et al. J Clin Invest 128, 3558-3567, doi: 10.1172/JC199081 (2018)), and tau to treat AD and Frontotemporal Dementia (FTD) (DeVos et al. Sci Transl Med 9, doi: 10.1126/scitranslmed.aag0481 (2017).; NCT03186989).

ASOs need to be delivered by continuous infusion or repetitive intrathecal injections into the cerebrospinal fluid, and their therapeutic effect is thought to be most potent in the brain areas adjacent to the ventricular system (Kordasiewicz et al. Neuron 74, 1031-1044, doi: 10.1016/j.neuron.2012.05.009 (2012)). While ASO therapies are promising, caveats of these treatments include the requirement for repeated administration and the continued cost of the drug over the lifetime of the patient. In humans, current ASO treatments require intrathecal injection every 3-4 months, which will build up scar tissue over time and may pose additional risk for infection in aged populations. Alternatively, RNAi-based gene therapy approaches using short hairpin RNA (shRNA) and microRNA (miRNA) are also being developed for neurodegenerative diseases. RNAi-based gene therapy uses a single administration of a viral vector that results in continuous expression of shRNA or artificial miRNA precursors and subsequent long-lasting lowering of the target protein. Artificial miRNAs mimic natural miRNA structures and can be engineered to silence potentially any gene of interest by base-pairing to complementary sequences on target mRNAs. Gene therapy studies using vector-mediated delivery of a miRNA shuttles have shown promise for treating HD in preclinical models and have recently reached the clinic (Evers et al. Mol Ther26, 2163-2177, doi: 10.1016/j.ymthe.2018.06.021 (2018): NCT04120493).

For gene delivery, AAV has emerged as one of the safest and most commonly used vectors due to their ability to infect non-dividing cells, high transduction efficiency, long-lasting expression from a single dose, and a relatively low host immune response. Recombinant AAV (rAAV) vectors retain only the inverted terminal repeats of wild-type virus, which flank a transgene cassette that consists of a promoter with the gene of interest to be delivered.

Advances in AAV vector design and delivery in the brain and spinal cord have made AAV ideal for the treatment of neurological diseases with the AAV9 serotype having the highest tropism for the CNS (Kantor Adv Genet 87, 125-197, doi: 10.1016/B978-0-12- 800149-3.00003-2 (2014)).

IND-enabling studies for Giant Axonal Neuropathy to support a Phase I clinical trial in children using an AAV9 vector administered intrathecally have been conducted (NCT02362438), which has been ongoing since 2015 (Bailey et al., Mol Ther Methods Clin Dev 9, 160-171, doi:10.1016/j.omtm.2018.02.005 (2018)). In addition to GAN, AAV9 gene therapy is being used in ongoing clinical trials for multiple disorders and the FDA recently approved the use of an AAV9 vector therapy for Spinal Muscular Atrophy.

Currently there are no approved therapies for neurodegenerative disorders that significantly slow disease progression and there remains a great need for disease-specific therapeutics.

### SUMMARY

Provided herein is an rAAV vector comprising at least one polynucleotide sequence encoding at least one amiRNA directed against MAPT, wherein the amiRNA directed against MAPT comprises the nucleic acid sequence set forth in any one of SEQ ID NOs: 139-186. In some embodiments, the polynucleotide sequence encoding the at least one amiRNA directed against MAPT comprises the nucleic acid sequence set forth in any one of SEQ ID NOs: 41-88. In some embodiments, the amiRNA is direct against human MAPT. In some embodiments, the amiRNA directed against human MAPT comprises the nucleic acid sequence set forth in any one of SEQ ID NOs: 139-149. In some embodiments, the amiRNA directed against human MAPT encoded by polynucleotide comprising the nucleic acid sequence set forth in any one of SEQ ID NO: 41-49.

In some embodiments, the rAAV vector further comprises a first inverted terminal repeat (ITR) sequence, wherein the first ITR sequence is an AAV2 ITR sequence. In some embodiments, the first ITR sequence comprises the sequence set forth in SEQ ID NO: 15. In some embodiments, the rAAV vector further comprises a second ITR sequence, wherein the second ITR sequence is an AAV2 ITR sequence. In some embodiments, the second ITR sequence comprises the sequence set forth in SEQ ID NO: 16.

In some embodiments, the rAAV vector further comprises a first promoter sequence, wherein the first promoter sequence is a murine U6 promoter sequence. In some embodiments, the murine U6 promoter sequence comprises the nucleic acid sequence set forth in SEQ ID NO: 31.

In some embodiments, the rAAV vector further comprises a second promoter sequence, wherein the second promoter sequence is a CBh promoter sequence. In some embodiments, the CBh promoter sequence comprises the nucleic acid sequence set forth in SEQ ID NO: 29.

In some embodiments, the rAAV vector further comprises a termination signal. In some embodiments, the termination signal comprises the nucleic acid sequence set forth in SEQ ID NO: 40.

In another aspect, provided herein is an rAAV vector comprising, in the 5′ to 3′ direction a first AAV2 ITR sequence, a murine U6 promoter sequence, a polynucleotide sequence encoding for at least one amiRNA directed against MAPT, a termination sequence, a CBh promoter sequence, a synthetic polyA sequence, and a second AAV2 ITR sequence, wherein the amiRNA directed against MAPT comprises the nucleic acid sequence set forth in any one of SEQ ID NOs: 139-186. In some embodiments, the rAAV vector comprises the sequence set forth in SEQ ID NO: 14.

In another aspect, provided herein is an rAAV viral vector comprising an AAV capsid protein: and an rAAV vector described herein. In some embodiments, the AAV capsid protein is an AAV9 capsid protein.

In another aspect, provided herein is a pharmaceutical composition comprising an rAAV vector described herein or an rAAV viral vector described herein and at least one pharmaceutically acceptable excipient and/or additive.

In another aspect, provided herein is a method for treating a tauopathy in a subject in need thereof, the method comprising administering to the subject a therapeutically effective amount of an rAAV vector, an rAAV viral vector, or a pharmaceutical composition described herein. In some embodiments, the tauopathy is Alzheimer's Disease or FTDP-17. In some embodiments, the subject has one or more mutations in the MAPT gene.

In another aspect, provided herein is an rAAV vector, an rAAV viral vector, or a pharmaceutical composition described herein for use in the treatment of a tauopathy. In some embodiments, the tauopathy is Alzheimer's Disease or FTDP-17. In some embodiments, the tauopathy is associated with one or more mutations in the MAPT gene.

### BRIEF DESCRIPTION OF THE DRAWINGS

The above and further features will be more clearly appreciated from the following detailed description when taken in conjunction with the accompanying drawings.

FIG. 1 shows a schematic of a self-complementary AAV9 vector comprising an anti-tau miRNA driven by a U6 promoter.

FIG. 2 shows knockdown of human MAPT various miRNAs directed against human Tau as measured by a dual reporter assay: scr=scrambled tau miRNA: hTau=human-specific tau miRNA.

FIGS. 3A and 3B show knockdown of human Tau mRNA and protein, respectively, with various miRNAs directed against human Tau.

FIGS. 3C and 3D show Tau expression in HEK293 cells after treatment with scrambled control miRNA and miRNA specific to human Tau, respectively, as determined by immunofluorescence.

FIG. 4A shows vector distribution by GFP staining in glia and neurons and a corresponding decrease of tau protein with either vehicle control or an AAV9 vector expressing miRNA directed against human Tau and also encoding a GFP reporter protein after delivery in mice overexpressing human P301S mutant tau.

FIG. 4B shows a quantification of MAPT mRNA in the treated mouse brain.

FIG. 5 shows knockdown of mouse Mapt with various miRNAs directed against mouse Tau as measured by a dual reporter assay: scr=scrambled tau miRNA: mTau=mouse-specific tau miRNA.

FIG. 6A shows knockdown of mouse Mapt mRNA with various miRNAs directed against mouse Tau.

FIG. 6B shows knockdown of mouse, but not human Tau protein with various miRNAs directed against mouse Tau.

FIGS. 7A and 7B show knockdown of mouse Mapt and human MAPT, respectively, with various miRNAs directed against both human and mouse Tau.

FIG. 8 shows survival of wildtype mice and mice overexpressing human P301S mutant tau treated with vehicle control or AAV9/hTau5i-GFP at 3 months of age.

FIGS. 9A and 9B show changes in body weight of wildtype mice and mice overexpressing human P301S mutant tau treated with vehicle control or AAV9/hTau5i-GFP (data for male and female mice shown in FIG. 9A and FIG. 9B, respectively).

FIG. 10 shows representative images of immunohistochemical staining using a GFP antibody in mice brains after ICM injection of AAV9/hTau5i-GFP or vehicle.

FIG. 11 shows MAPT expression as determined by qPCR in brainstem tissue from wildtype mice and mice overexpressing human P301S mutant tau treated with vehicle control or AAV9/hTauSi-GFP.

FIGS. 12A-12C show Tau expression as determined by ELISA in brainstem, cerebellum and cortex tissue from wildtype mice and mice overexpressing human P301S mutant tau treated with vehicle control or AAV9/hTau5i-GFP.

FIGS. 13A-13C show seeding assay results where transfection of brain homogenate from wildtype mice treated with vehicle or AAV9/hTau5i-GFP resulted in no FRET-positive inclusions and the addition of brain lysate from P301S treatment cohorts induced intracellular FRET positive aggregates and that P301S mice treated with AAV9/hTauSi-GFP induce less FRET positive aggregates as compared to vehicle treated P301S mice. (One-way ANOVA, Dunnett's multiple comparison test compared to P301S+Vehicle, ****p<0.0001).

FIG. 14 shows survival of wildtype mice and mice overexpressing human P301S mutant tau treated with vehicle control, AAV9/Scrambled (Scr) control, AAV9/hTau5i-GFP or AAV9/hTauSi at 6 months of age.

FIGS. 15A and 15B show changes in body weight of wildtype mice and mice overexpressing human P301S mutant tau treated with vehicle control, AAV9/Scr control, AAV9/hTauSi-GFP or AAV9/hTauSi at 6 months of age.

FIGS. 16A-16D shows seeding assay results. FIGS. 16A and 16B show that AAV9/Scr did not change seeding activity in WT or P301S mice as compared to vehicle treatment. FIG. 16C shows that mice treated with AAV9/hTau5i-GFP or AAV9/hTau5i had similar seeding activity in brainstem. FIG. 16D shows that treatment with AAV9/hTauSi at 6 months of age significantly reduced tau seeding activity in the brainstem.

FIG. 17 shows survival of wildtype mice and mice overexpressing human P301S mutant tau treated with AAV9/Scr control or AAV9/hTau5i at 9 months of age.

FIGS. 18A and 18B show shows changes in body weight of wildtype mice and mice overexpressing human P301S mutant tau treated with AAV9/Scr control or AAV9/hTau5i at 9 months of age.

FIGS. 19A-19E show no changes in clinical blood chemistry 1 month-post injection from wildtype mice treated with vehicle, AAV9/Scr control or AAV9/mTau2i at 3 months of age.

FIGS. 20A-20C show no change in behavior 1 month-post injection from wildtype mice treated with vehicle, AAV9/Scr control or AAV9/mTau2i at 3 months of age.

### DETAILED DESCRIPTION

The present disclosure provides, inter alia, isolated nucleic acid molecules, recombinant adeno-associated virus (rAAV) vectors, and rAAV viral vectors comprising at least one polynucleotide sequence encoding at least one amiRNA molecule directed against MAPT. The present disclosure also provides methods of manufacturing these isolated polynucleotides, rAAV vectors, and rAAV viral vectors, as well as their use to deliver shRNA molecules to treat or prevent tauopathies.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.: and Rose, Comprehensive Virology 3: 1-61 (1974)). For example, all AAV serotypes apparently exhibit very similar replication properties mediated by homologous rep genes: and all bear three related capsid proteins such as those expressed in AAV2. The degree of relatedness is further suggested by heteroduplex analysis which reveals extensive cross-hybridization between serotypes along the length of the genome: and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery: all known serotypes can infect cells from various tissue types. At least 11 sequentially numbered AAV serotypes are known in the art. Non-limiting exemplary serotypes useful in the methods disclosed herein include any of the 11 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In some aspects, the AAV refers to the serotype AAVI, AAV2, AAV4, AAV5, AAV6, AAV7, AAV8, AAV9, AAV10, AAV11, AAV12, AAV13, AAVPHP.B. AAVrh74 or AA Vrh. 10.

Exemplary adeno-associated viruses and recombinant adeno-associated viruses include, but are not limited to all serotypes (e.g., AAVI, AAV2, AAV3, AAV4, AAV5, AAV6, AAV7, AAV8, AAV9, AAV10, AAV11, AAV12, AAV13, AAVPHP.B, AAVrh74 and AAVrh. 10). Exemplary adeno-associated viruses and recombinant adeno-associated viruses include, but are not limited to, self-complementary AAV (scAAV) and AAV hybrids containing the genome of one serotype and the capsid of another serotype (e.g., AAV2/5, AAV-DJ and AAV-DJ8). Exemplary adeno-associated viruses and recombinant adeno-associated viruses include, but are not limited to, rAAV-LK03, AAV-KP-1 (described in detail in Kerun et al. JCI Insight, 2019: 4(22):e131610) and AAV-NP59 (described in detail in Paulk et al. Molecular Therapy, 2018: 26(1): 289-303).

### AAV Structure and Function

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively: the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra SP et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid genc: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe JP, Carter BJ (September 1988). “Alternate mRNA splicing is required for synthesis of adeno-associated virus VP I capsid protein” Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate rAAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. Sec, Clark et al., Hum Gene Ther, 8: 659-669 (1997): Kessler et al., Proc Nat Acad Sc USA, 93: 14082-14087 (1996); and Xiao et al., J Virol, 70: 8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94: 5804-5809 (1997) and Murphy et al., Proc Natl Acad Sci USA, 94: 13921-13926 (1997). Moreover, Lewis et al., J Virol, 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV (rAAV) genomes of the invention comprise, consist essentially of, or consist of a nucleic acid molecule comprising a polynucleotide sequencing encoding at least one amiRNA directed against MAPT and one or more AAV ITRs flanking the nucleic acid molecule. Production of pseudotyped rAAV is disclosed in, for example, WO2001083692. Other types of rAAV variants, for example rAAV with capsid mutations, are also contemplated. See. e.g., Marsic et al., Molecular Therapy, 22(11): 1900-1909 (2014). The nucleotide sequences of the genomes of various AAV serotypes are known in the art.

### Isolated Nucleic Acid Molecules

The present disclosure provides isolated nucleic acid molecules comprising a polynucleotide sequence encoding at least one amiRNA directed against MAPT.

Illustrative polynucleotide sequences encoding amiRNA sequences directed against human MAPT (“shuttle DNA”), as well as the corresponding amiRNA sequences directed against human MAPT are set forth in Table 1. Illustrative polynucleotide sequences encoding amiRNA sequences directed against mouse MAPT are set forth in SEQ ID NOs: 52-70 and the corresponding amiRNA sequences directed against mouse MAPT are set forth in SEQ ID NOs: 150-168. Illustrative polynucleotide sequences encoding amiRNA sequences directed against mouse and human MAPT are set forth in SEQ ID NOs: 71-88 and the corresponding amiRNA sequences directed against mouse and human MAPT are set forth in SEQ ID NOs: 169-168.

TABLE 1
Exemplary Polynucleotide Sequences Encoding amiRNAs Directed Against MAPT

| miRNA | Shuttle DNA (5′-3′) | amiRNA Sequence (5′-3′) |
|---|---|---|
| hTau1i | GAGTGAGCGACCGCCAGGAGTTCGAAGTGA | AUCACUUCGAACUCCUGG |
| | TCTGTAAAGCCACAGATGGGATCACTTCGAA | CGGG (SEQ ID NO: 139) |
| | CTCCTGGCGGGTGCCTACT (SEQ ID NO: 41) | |
| hTau2i | GAGTGAGCGCCGCTGGCCTGAAAGCTGAAG | UCUUCAGCUUUCAGGCCA |
| | ACTGTAAAGCCACAGATGGGTCTTCAGCTTT | GCGU (SEQ ID NO: 140) |
| | CAGGCCAGCGTTGCCTACT (SEQ ID NO: 42) | |
| hTau3i | GAGTGAGCGCCGGGACTGGAAGCGATGACA | UUGUCAUCGCUUCCAGUC |
| | ACTGTAAAGCCACAGATGGGTTGTCATCGCT | CCGU (SEQ ID NO: 141) |
| | TCCAGTCCCGTTGCCTACT (SEQ ID NO: 43) | |
| hTau4i | GAGTGAGCGATGGCGGAGGAAATAAAAAGA | AUCUUUUUAUUUCCUCCG |
| | TCTGTAAAGCCACAGATGGGATCTTTTTATTT | CCAG (SEQ ID NO: 142) |
| | CCTCCGCCAGTGCCTACT (SEQ ID NO: 44) | |
| hTau5i | GAGTGAGCGCCGCCACCAGGATTCCAGCAA | UUUGCUGGAAUCCUGGU |
| | ACTGTAAAGCCACAGATGGGTTTGCTGGAAT | GGCGU (SEQ ID NO: 143) |
| | CCTGGTGGCGTTGCCTACT (SEQ ID NO: 45) | |
| hTau6i | GAGTGAGCGCTCGCATGGTCAGTAAAAGCA | UUGCUUUUACUGACCAU |
| | ACTGTAAAGCCACAGATGGGTTGCTTTTACT | GCGAG (SEQ ID NO: 144) |
| | GACCATGCGAGTGCCTACT (SEQ ID NO: 46) | |
| hTau7i | GAGTGAGCGAGAGTTCGAAGTGATGGAAGA | AUCUUCCAUCACUUCGAA |
| | TCTGTAAAGCCACAGATGGGATCTTCCATCA | CUCC (SEQ ID NO: 145) |
| | CTTCGAACTCCTGCCTACT (SEQ ID NO: 47) | |
| hTau8i | GAGTGAGCGCCCCAAGCTCGCATGGTCAGTA | UACUGACCAUGCGAGCUU |
| | CTGTAAAGCCACAGATGGGTACTGACCATGC | GGGU (SEQ ID NO: 146) |
| | GAGCTTGGGTTGCCTACT (SEQ ID NO: 48) | |
| hTau9i | GAGTGAGCGCGCCAAGGGGGCTGATGGTAA | UUUACCAUCAGCCCCCUU |
| | ACTGTAAAGCCACAGATGGGTTTACCATCAG | GGCU (SEQ ID NO: 147) |
| | CCCCCTTGGCTTGCCTACT (SEQ ID NO: 49) | |
| hTau10i | GAGTGAGCGACGGCAGCATCGACATGGTAG | UCUACCAUGUCGAUGCUG |
| | ACTGTAAAGCCACAGATGGGTCTACCATGTC | CCGG (SEQ ID NO: 148) |
| | GATGCTGCCGGTGCCTACT (SEQ ID NO: 50) | |
| hTau11i | GAGTGAGCGCCGGGGCGGAGATCGTGTACA | UUGUACACGAUCUCCGCC |
| | ACTGTAAAGCCACAGATGGGTTGTACACGAT | CCGU (SEQ ID NO: 149) |
| | CTCCGCCCCGTTGCCTACT (SEQ ID NO: 51) | |

In some embodiments, the amiRNA directed against MAPT comprises, consists essentially of, or consists of the nucleic acid sequence set forth in any one of SEQ ID NOs: 139-186. In some embodiments, the amiRNA directed against MAPT comprises, consists essentially of, or consists of a nucleic acid sequence that is at least 65%, 70%, 75%, 80%, 85%, 90%, 95%, 96%, 97%, 98%, 99% or 100% (or any percentage in between) identical to the nucleic acid sequence set forth in any one of SEQ ID NOs: 139-186.

In some embodiments, the polynucleotide sequence encoding the amiRNA directed against MAPT comprises, consists essentially of, or consists of the nucleic acid sequence set forth in any one of SEQ ID NOs: 41-88. In some embodiments, the polynucleotide sequence encoding the amiRNA directed against MAPT comprises, consists essentially of, or consists of a nucleic acid sequence that is at least 65%, 70%, 75%, 80%, 85%, 90%, 95%, 96%, 97%, 98%, 99% or 100% (or any percentage in between) identical to the nucleic acid sequence set forth in any one of SEQ ID NOs: 41-88. SEQ ID NOs: 41-88 are DNA sequences and the corresponding RNA sequences are set forth in SEQ ID NOs: 90-137, respectively.

Generally, a polynucleotide sequence encoding the amiRNA directed against MAPT provided herein comprises an amiRNA directed against MAPT (a mature antisense sequence), a loop sequence, and a passenger sequence, flanked by 5′ and 3′ overhangs.

The loop sequence and overhangs together make up the miRNA “backbone.” An exemplary loop sequence is CTGTAAAGCCACAGATGGG (DNA, SEQ ID NO: 188) or CUGUAAAGCCACAGAUGGG (RNA, SEQ ID NO: 189). An exemplary 5′ overhang sequence is GAGTGAGCG (DNA, SEQ ID NO: 190) or GAGUGAGCG (RNA, SEQ ID NO: 191). An exemplary 3′ overhang sequence is TGCCTACT (DNA, SEQ ID NO: 192) or UGCCUACU (RNA, SEQ ID NO: 193). Without wishing to be bound by theory, it is believed that the passenger sequence and the amiRNA directed against MAPT adhere to form a hairpin loop structure. The hairpin loop structures are then believed to be further processed by Dicer, an endoribonuclease which removes the loop of the hairpin, leaving a miRNA duplex. The amiRNA directed against MAPT of the miRNA duplex is then integrated into the RNA-induced silencing complex (RISC), where it interacts with its target mRNA (e.g., MAPT mRNA) and thus blocks translation. See Myburgh et al. Molecular Therapy—Nucleic Acids (2014) 3, c207.

The loop and overhang sequences form a miRNA scaffold. It will be apparent to a person of skill in the art that any suitable scaffold may be used to deliver the amiRNAs directed against MAPT provided herein. Exemplary scaffolds include the miR-30 scaffold (Chang et al., Cold Spring Harb Protoc: 2013: doi: 10.1101/pdb.prot075853), and the miR-33 scaffold (Xie et al., 2020 Mor Ther. 28(2):422), The amiRNAs directed against MAPT may be cloned into any suitable miR scaffold, as would be appreciated by the skilled artisan.

In some embodiments, the MAPT transcript against an amiRNA described herein is directed is a human MAPT transcript. Illustrative human MAPT transcripts against which the amiRNAs provided herein may be directed include those set forth in NCBI Reference Sequences NM_016835.5, NM_005910.6, NM_016834.5, NM_016841.5, NM_001123067.4, NM_001123066.4, NM_001203251.2. NM_001203252.2, NM_001377265.1, NM_001377266.1, NM_001377267.1, and NM_001377268.1 (SEQ ID NOs: 2-13, respectively). In some embodiments, amiRNA described herein targets 1, 2, 3, 4, 5, or 6 MAPT transcripts. In some embodiments, an amiRNA described herein targets a human MAPT transcript. In some embodiments, amiRNA described herein targets 1, 2, 3, 4, 5, or 6 human MAPT transcripts.

As would be appreciated by the skilled artisan, the phrase “amiRNA directed against MAPT” refers to an RNA molecule that, once produced within a cell, directs endogenous RNAi pathways (e.g. the Dicer pathway, the RNA-induced silencing complex (RISC) pathway) to initiate degradation and/or downregulation of MAPT mRNA. The term “MAPT targeting AAV-amiRNA” refers to an rAAV vector comprising at least one polynucleotide sequence encoding an amiRNA directed against MAPT.

In some aspects, an isolated nucleic acid molecule can comprise more than one polynucleotide sequence encoding an amiRNA directed against MAPT. Thus, in some embodiments, an isolated nucleic acid molecules comprises at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20 polynucleotide sequences, each encoding an amiRNA directed against MAPT.

In aspects wherein an isolated nucleic acid molecule comprises more than one polynucleotide sequence encoding an amiRNA directed against MAPT, any number of the polynucleotide sequences may be the same sequence, and any number may be a different sequence. In a non-limiting example wherein an isolated nucleic acid molecule comprises three polynucleotide sequences each encoding an amiRNA directed against MAPT (i.e. a first polynucleotide sequence, a second polynucleotide sequence, and a third polynucleotide sequence) all three of the polynucleotide sequences can have the same sequence. Alternatively, all three of the polynucleotide sequences can have a different sequence. Alternatively still, two of the three polynucleotide sequences can have the same sequence and the third polynucleotide sequence can have a different sequence.

rAAV Vectors

In some aspects, the isolated polynucleotides comprising at least one polynucleotide sequence encoding at least one amiRNA directed against MAPT described herein can be a recombinant AAV (rAAV) vector.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episomal) element or may be integrated into a host cell chromosome. Vectors may include nucleic acids derived from retroviruses, adenoviruses, herpesvirus, baculoviruses, modified baculoviruses, papovaviruses, or otherwise modified naturally-occurring viruses. Exemplary non-viral vectors for delivering nucleic acid include naked DNA: DNA complexed with cationic lipids, alone or in combination with cationic polymers: anionic and cationic liposomes: DNA-protein complexes and particles comprising, consisting essentially of, or consisting of DNA condensed with cationic polymers such as heterogeneous polylysine, defined-length oligopeptides, and polyethyleneimine, in some cases contained in liposomes; and the use of ternary complexes comprising, consisting essentially of, or consisting of a virus and polylysine-DNA.

With respect to general recombinant techniques, vectors that contain both a promoter and a cloning site into which a polynucleotide can be operatively linked are well known in the art. Such vectors are capable of transcribing RNA in vitro or in vivo, and are commercially available from sources such as Agilent Technologies (Santa Clara, Calif) and Promega Biotech (Madison, Wis.). In order to optimize expression and/or in vitro transcription, it may be necessary to remove, add or alter 5′ and/or 3′ untranslated portions of cloned transgenes to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. The nucleic acid sequence of the AAV vector may be codon-optimized.

An “rAAV vector” as used herein refers to a vector comprising, consisting essentially of, or consisting of one or more transgene and/or exogenous polynucleotide sequences and one or more AAV inverted terminal repeat sequences (ITRs). Such rAAV vectors can be replicated and packaged into infectious viral particles when present in a host cell that provides the functionality of rep and cap gene products: for example, by transfection of the host cell. In some aspects, rAAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the rAAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least one polynucleotide sequence encoding at least one amiRNA directed against MAPT. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence.

In some aspects, an rAAV vector can comprise a first AAV ITR sequence, a promoter sequence, at least one polynucleotide sequence encoding at least one amiRNA directed against MAPT and a second AAV ITR sequence. In some aspects, an rAAV vector can comprise, in the 5′ to 3′ direction, a first AAV ITR sequence, a promoter sequence, at least one polynucleotide sequence encoding at least amiRNA directed against MAPT and a second AAV ITR sequence.

In some aspects, an rAAV vector can comprise a first AAV ITR sequence, a promoter sequence, at least one polynucleotide sequence encoding at least one amiRNA directed against MAPT, a polyA sequence, and a second AAV ITR sequence. In some aspects, an rAAV vector can comprise, in the 5′ to 3′ direction, a first AAV ITR sequence, a promoter sequence, at least one polynucleotide sequence encoding at least one amiRNA directed against MAPT, a polyA sequence, and a second AAV ITR sequence.

In some aspects, an rAAV vector can comprise more than one polynucleotide sequence encoding at least one amiRNA directed against MAPT.

In some aspects, the present disclosure provides rAAV vectors comprising at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20 polynucleotide sequences, each encoding at least one amiRNA directed against MAPT.

In some aspects, the present disclosure provides rAAV vectors comprising about two, or about three, or about four, or about five, or about six, or about seven, or about eight, or about nine, or about ten, or about 11, or about 12, or about 13, or about 14, or about 15, or about 16, or about 17, or about 18, or about 19, or about 20 polynucleotide sequences, each encoding at least one amiRNA directed against MAPT.

In aspects wherein an rAAV vector comprises more than one polynucleotide sequence encoding at least one amiRNA directed against MAPT, any number of the polynucleotide sequences may be the same sequence, and any number may be a different sequence. In a non-limiting example wherein an rAAV vector comprises three polynucleotide sequences encoding at least one amiRNA directed against MAPT (i.e. a first polynucleotide sequence, a second polynucleotide sequence, and a third polynucleotide sequence), all three of the polynucleotide sequences can have the same sequence. Alternatively, all three of the polynucleotide sequences can have a different sequence. Alternatively still, two of the three polynucleotide sequences can have the same sequence and the third polynucleotide sequence can have a different sequence.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first polynucleotide sequence encoding at least one amiRNA directed against MAPT and an at least second polynucleotide sequence encoding at least one amiRNA directed against MAPT, the first promoter can be located upstream (5′) of the first polynucleotide sequence and the at least second promoter can be located between the first polynucleotide sequence and the at least second polynucleotide sequence, such that the at least second promoter is downstream (3′) of the first polynucleotide sequence and upstream (5′) of the at least second polynucleotide sequence.

Any of the preceding rAAV vectors can further comprise at least one enhancer. The at least one enhancer can be located anywhere in the rAAV vector.

In some aspects, the at least one enhancer can be located immediately upstream (5′) of a promoter. Thus, an rAAV vector can comprise, in the 5′ to 3′ direction, a first AAV ITR sequence, an enhancer, a promoter sequence, at least one polynucleotide sequence encoding at least one amiRNA directed against MAPT and a second AAV ITR sequence. In some aspects, the at least one enhancer can be located immediately downstream (3′) of a promoter. Thus, an rAAV vector can comprise, in the 5′ to 3′ direction, a first AAV ITR sequence, a promoter sequence, an enhancer, at least one polynucleotide sequence encoding at least one amiRNA directed against MAPT, and a second AAV ITR sequence. In some aspects, the at least one enhancer can be located immediately downstream of an at least one polynucleotide sequence encoding at least one amiRNA directed against MAPT. Thus, an rAAV vector can comprise, in the 5′ to 3′ direction, a first AAV ITR sequence, a promoter sequence, at least one polynucleotide sequence encoding at least one amiRNA directed against MAPT, an enhancer, a polyA sequence, and a second AAV ITR sequence.

In some embodiments, an rAAV vector comprises a mutant AAV2 ITR with the D element deleted, a U6 promoter, an polynucleotide sequence encoding an amiRNA directed against MAPT, a T6 termination signal, a CBh promoter, a polyadenylation signal, and wild-type AAV2 ITR.

An illustrative sequences of an rAAV vector comprising a polynucleotide encoding an amiRNA directed against MAPT is set forth in SEQ ID NO: 14. ITR sequences are underlined in SEQID NO: 14: the promoter region is italicized, the sequence encoding hTau5i is shown in bold-italics.

CTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGGGTTCGGT
ACCCCTAGGGACGCCGCCATCTCTAGGCCCGCGCCGGCCCCCTCGCACAGACTTGTGGGAGA
AGCTCGGCTACTCCCCTGCCCCGGTTAATTTGCATATAATATTTCCTAGTAACTATAGAGGCTTA
ATGTGCGATAAAAGACAGATAATCTGTTCTTTTTAATACTAGCTACATTTTACATGATAGGCTTGG
ATTTCTATAAGAGATACAAATACTAAATTATTATTTTAAAAAACAGCACAAAAGGAAACTCACCCTA
ACTGTAAAGTAATTGTGTGTTTTGAGACTATAAATATCCCTTGGAGAAAAGCCTTGTTTGCGTTT
AGTGAACCGTCAGATGGTACCGTTTAAACTCGAGTGAGCGCCGCCACCAGGATTCCAGCA
AACTGTAAAGCCACAGATGGGTTTGCTGGAATCCTGGTGGCGTTGCCTACTAGAGCGGCCG
CCACAGCGGGGAGATCCAGACATGATAAGATACATTTTTTGAATTCAGATCCGAGCTCGG
TACCAAGCTTGGTACCCGTTACATAACTTACGGTAAATGGCCCGCCTGGCTGACCGCCCA
ACGACCCCCGCCCATTGACGTCAATAGTAACGCCAATAGGGACTTTCCATTGACGTCAAT
GGGTGGAGTATTTACGGTAAACTGCCCACTTGGCAGTACATCAAGTGTATCATATGCCAA
GTACGCCCCCTATTGACGTCAATGACGGTAAATGGCCCGCCTGGCATTGTGCCCAGTACA
TGACCTTATGGGACTTTCCTACTTGGCAGTACATCTACGTATTAGTCATCGCTATTACCAT
GGTCGAGGTGAGCCCCACGTTCTGCTTCACTCTCCCCATCTCCCCCCCCTCCCCACCCCCA
ATTTTGTATTTATTTATTTTTTAATTATTTTGTGCAGCGATGGGGGCGGGGGGGGGGGGGG
GGCGCGCGCCAGGCGGGGCGGGGCGGGGCGAGGGGCGGGGCGGGGCGAGGCGGAGAGG
TGCGGCGGCAGCCAATCAGAGCGGCGCGCTCCGAAAGTTTCCTTTTATGGCGAGGCGGCG
GCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCG
CGTTACTCCCACAGGTGAGCGGGCGGGACGGCCCTTCTCCTCCGGGCTGTAATTAGCTGA
GCAAGAGGTAAGGGTTTAAGGGATGGTTGGTTGGTGGGGTATTAATGTTTAATTACCTGG
AGCACCTGCCTGAAATCACTTTTTTTCAGGTTGGACCTAATAAAGAGCTCAGATGCATCG
ATCAGAGTGTGTTGGTTTTTTGTGTGACGCGTAGGAACCCCTAGTGATGGAGTTGGCCAC
TCCCTCTCTGCGCGCTCGCTCGCTCACTGAGGCCGGGCGACCAAAGGTCGCCCGACGCCC
GGGCTTTGCCCGGGCGGCCTCAGTGAGCGAGCGAGCGCGCAGCTGGCGTAATAGCGAAG
AGGCCCGCACCGATCGCCCTTCCCAACAGTTGCGCAGCCTGAATGGCGAATGGCGATTCC
GTTGCAATGGCTGGCGGTAATATTGTTCTGGATATTACCAGCAAGGCCGATAGTTTGAGT
TCTTCTACTCAGGCAAGTGATGTTATTACTAATCAAAGAAGTATTGCGACAACGGTTAAT
TTGCGTGATGGACAGACTCTTTTACTCGGTGGCCTCACTGATTATAAAAACACTTCTCAGG
ATTCTGGCGTACCGTTCCTGTCTAAAATCCCTTTAATCGGCCTCCTGTTTAGCTCCCGCTCT
GATTCTAACGAGGAAAGCACGTTATACGTGCTCGTCAAAGCAACCATAGTACGCGCCCTG
TAGCGGCGCATTAAGCGCGGCGGGTGTGGTGGTTACGCGCAGCGTGACCGCTACACTTGC
CAGCGCCCTAGCGCCCGCTCCTTTCGCTTTCTTCCCTTCCTTTCTCGCCACGTTCGCCGGCT
TTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATA
GACGGTTTTTCGCCCTTTGACGTTGGAGTCCACGTTCTTTAATAGTGGACTCTTGTTCCAA
ACTGGAACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTTTAACA
AAATATTAACGCTTACAATTTAAATATTTGCTTATACAATCTTCCTGTTTTTGGGGCTTTTC
TGATTATCAACCGGGGTACATATGATTGACATGCTAGTTTTACGATTACCGTTCATCGATT
CTCTTGTTTGCTCCAGACTCTCAGGCAATGACCTGATAGCCTTTGTAGAGACCTCTCAAAA
ATAGCTACCCTCTCCGGCATGAATTTATCAGCTAGAACGGTTGAATATCATATTGATGGT
GATTTGACTGTCTCCGGCCTTTCTCACCCGTTTGAATCTTTACCTACACATTACTCAGGCA
TTGCATTTAAAATATATGAGGGTTCTAAAAATTTTTATCCTTGCGTTGAAATAAAGGCTTC
TCCCGCAAAAGTATTACAGGGTCATAATGTTTTTGGTACAACCGATTTAGCTTTATGCTCT
GAGGCTTTATTGCTTAATTTTGCTAATTCTTTGCCTTGCCTGTATGATTTATTGGATGTTGG
AATCGCCTGATGCGGTATTTTCTCCTTACGCATCTGTGCGGTATTTCACACCGCATATGGT
GCACTCTCAGTACAATCTGCTCTGATGCCGCATAGTTAAGCCAGCCCCGACACCCGCCAA
CACCCGCTGACGCGCCCTGACGGGCTTGTCTGCTCCCGGCATCCGCTTACAGACAAGCTG
TGACCGTCTCCGGGAGCTGCATGTGTCAGAGGTTTTCACCGTCATCACCGAAACGCGCGA
GACGAAAGGGCCTCGTGATACGCCTATTTTTATAGGTTAATGTCATGATAATAATGGTTT
CTTAGACGTCAGGTGGCACTTTTCGGGGAAATGTGCGCGGAACCCCTATTTGTTTATTTTT
CTAAATACATTCAAATATGTATCCGCTCATGAGACAATAACCCTGATAAATGCTTCAATA
ATATTGAAAAAGGAAGAGTATGAGCCATATTCAACGGGAAACGTCTTGCTCTAGGCCGC
GATTAAATTCCAACATGGATGCTGATTTATATGGGTATAAATGGGCTCGCGATAATGTCG
GGCAATCAGGTGCGACAATCTATCGATTGTATGGGAAGCCCGATGCGCCAGAGTTGTTTC
TGAAACATGGCAAAGGTAGCGTTGCCAATGATGTTACAGATGAGATGGTCAGACTAAAC
TGGCTGACGGAATTTATGCCTCTTCCGACCATCAAGCATTTTATCCGTACTCCTGATGATG
CATGGTTACTCACCACTGCGATCCCTGGGAAAACAGCATTCCAGGTATTAGAAGAATATC
CTGATTCAGGTGAAAATATTGTTGATGCGCTGGCAGTGTTCCTGCGCCGGTTGCATTCGAT
TCCTGTTTGTAATTGTCCTTTTAACAGCGATCGCGTATTTCGTCTCGCTCAGGCGCAATCA
CGAATGAATAACGGTTTGGTTGATGCGAGTGATTTTGATGACGAGCGTAATGGCTGGCCT
GTTGAACAAGTCTGGAAAGAAATGCATAAACTTTTGCCATTCTCACCGGATTCAGTCGTC
ACTCATGGTGATTTCTCACTTGATAACCTTATTTTTGACGAGGGGAAATTAATAGGTTGTA
TTGATGTTGGACGAGTCGGAATCGCAGACCGATACCAGGATCTTGCCATCCTATGGAACT
GCCTCGGTGAGTTTTCTCCTTCATTACAGAAACGGCTTTTTCAAAAATATGGTATTGATAA
TCCTGATATGAATAAATTGCAGTTTCATTTGATGCTCGATGAGTTTTTCTAACTGTCAGAC
CAAGTTTACTCATATATACTTTAGATTGATTTAAAACTTCATTTTTAATTTAAAAGGATCT
AGGTGAAGATCCTTTTTGATAATCTCATGACCAAAATCCCTTAACGTGAGTTTTCGTTCCA
CTGAGCGTCAGACCCCGTAGAAAAGATCAAAGGATCTTCTTGAGATCCTTTTTTTCTGCG
CGTAATCTGCTGCTTGCAAACAAAAAAACCACCGCTACCAGCGGTGGTTTGTTTGCCGGA
TCAAGAGCTACCAACTCTTTTTCCGAAGGTAACTGGCTTCAGCAGAGCGCAGATACCAAA
TACTGTTCTTCTAGTGTAGCCGTAGTTAGGCCACCACTTCAAGAACTCTGTAGCACCGCCT
ACATACCTCGCTCTGCTAATCCTGTTACCAGTGGCTGCTGCCAGTGGCGATAAGTCGTGTC
TTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCT
ACAGCGTGAGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGGGAAACGC
CTGGTATCTTTATAGTCCTGTCGGGTTTCGCCACCTCTGACTTGAGCGTCGATTTTTGTGA
TGCTCGTCAGGGGGGCGGAGCCTATGGAAAAACGCCAGCAACGCGGCCTTTTTACGGTTC
CTGGCCTTTTGCTGGCCTTTTGCTCACATGTTCTTTCCTGCGTTATCCCCTGATTCTGTGGA
TAACCGTATTACCGCCTTTGAGTGAGCTGATACCGCTCGCCGCAGCCGAACGACCGAGCG
CAGCGAGTCAGTGAGCGAGGAAGCGGAAGAGCGCCCAATACGCAAACCGCCTCTCCCCG
CGCGTTGGCCGATTCATTAATGCAG

### AAV ITR Sequences

In some aspects, an AAV ITR sequence can comprise any AAV ITR sequence known in the art. In some aspects, an AAV ITR sequence can be an AAVI ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AAVrh74 ITR sequence or an AAVrh. 10 ITR sequence.

Thus, in some aspects, an AAV ITR sequence can comprise, consist essentially of, or consist of an AAVI ITR sequence, an AAV2 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAV11 ITR sequence, an AAV12 ITR sequence, an AAV13 ITR sequence, an AA Vrh74 ITR sequence, or an AAVrh. 10 ITR sequence.

In some aspects, an rAAV vector of the present disclosure can comprise, consist essentially of, or consist of AAV2 ITR sequences. In some aspects, an rAAV vector of the present disclosure can comprise, consist essentially of, or consist of AAV2 ITR sequences or a modified AAV2 ITR sequence. An AAV2 ITR may be modified, for example, by deleting the D region.

In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to any one of the nucleic acid sequences put forth in any one of SEQ ID NOs: 15-24, or complement thereof.

In some aspects, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NO: 15, or complement thereof.

In some aspects, a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the nucleic acid sequence set forth in SEQ ID NO: 16, or complement thereof.

### Promoter Sequence and Enhancers

The terms “promoter” and “promoter sequence” as used herein mean a control sequence that is a region of a polynucleotide sequence at which the initiation and rate of transcription of a coding sequence, such as a gene or a transgene or an shRNA sequence, are controlled. Promoters may be constitutive, inducible, repressible, or tissue-specific, for example. Promoters may contain genetic elements at which regulatory proteins and molecules such as RNA polymerase and transcription factors may bind. Non-limiting exemplary promoters include Rous sarcoma virus (RSV) LTR promoter (optionally with the RSV enhancer), a cytomegalovirus (CMV) promoter, an SV40 promoter, a dihydrofolate reductase promoter, a ß-actin promoter, a phosphoglycerol kinase (PGK) promoter, a U6 promoter, an H1 promoter, a ubiquitous chicken β-actin hybrid (CBh) promoter, a small nuclear RNA (U1a or U1b) promoter, an MeCP2 promoter, an MeP418 promoter, an MeP426 promoter, a minimal MeCP2 promoter, a VMD2 promoter, an mRho promoter, or an EF1 promoter. A promoter may be, for example, a human promoter sequence or a murine promoter sequence.

Additional non-limiting exemplary promoters provided herein include, but are not limited to EF1a, Ubc, human β-actin, CAG, TRE, Ac5, Polyhedrin, CaMKIIa, Ga11, TEF1, GDS, ADH1, Ubi, and α-1-antitrypsin (hAAT). It is known in the art that the nucleotide sequences of such promoters may be modified in order to increase or decrease the efficiency of mRNA transcription. See, e.g., Gao et al. (2018) Mol. Ther.: Nucleic Acids 12:135-145 (modifying TATA box of 7SK, U6 and H1 promoters to abolish RNA polymerase III transcription and stimulate RNA polymerase II-dependent mRNA transcription). Synthetically-derived promoters may be used for ubiquitous or tissue specific expression. Further, virus-derived promoters, some of which are noted above, may be useful in the methods disclosed herein, e.g., CMV, HIV, adenovirus, and AAV promoters. In some aspects, the promoter is used together with at least one enhancer to increase the transcription efficiency. Non-limiting examples of enhancers include an interstitial retinoid-binding protein (IRBP) enhancer, an RSV enhancer or a CMV enhancer.

In some aspects, a promoter sequence can comprise, consist essentially of, or consist of a Rous sarcoma virus (RSV) LTR promoter sequence (optionally with the RSV enhancer), a cytomegalovirus (CMV) promoter sequence, an SV40 promoter sequence, a dihydrofolate reductase promoter sequence, a β-actin promoter sequence, a phosphoglycerol kinase (PGK) promoter sequence, a human U6 promoter sequence, a mouse U6 promoter sequence, an H1 promoter sequence, a ubiquitous chicken β-actin hybrid (CBh) promoter sequence, a small nuclear RNA (U1a or U1b) promoter sequence, an MeCP2 promoter sequence, an MeP418 promoter sequence, an MeP426 promoter sequence, a meP229 promoter sequence, a minimal MeCP2 promoter sequence, a VMD2 promoter sequence, an mRho promoter sequence, an EFI promoter sequence, an EFIa promoter sequence, a Ubc promoter sequence, a human β-actin promoter sequence, a CAG promoter sequence, a TRE promoter sequence, an Ac5 promoter sequence, a Polyhedrin promoter sequence, a CaMKIIa promoter sequence, a Gal1 promoter sequence, a TEF1 promoter sequence, a GDS promoter sequence, an ADH1 promoter sequence, a Ubi promoter sequence or an α-1-antitrypsin (hAAT) promoter sequence.

An enhancer is a regulatory clement that increases the expression of a target sequence. A “promoter/enhancer” is a polynucleotide that contains sequences capable of providing both promoter and enhancer functions. For example, the long terminal repeats of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) or synthetic techniques such that transcription of that gene is directed by the linked enhancer/promoter. Non-limiting examples of linked enhancer/promoter for use in the methods, compositions and constructs provided herein include a PDE promoter plus IRBP enhancer or a CMV enhancer plus U1a promoter. It is understood in the art that enhancers can operate from a distance and irrespective of their orientation relative to the location of an endogenous or heterologous promoter. It is thus further understood that an enhancer operating at a distance from a promoter is thus “operably linked” to that promoter irrespective of its location in the vector or its orientation relative to the location of the promoter.

As used throughout the disclosure, the term “operably linked” refers to the expression of a polynucleotide sequence (i.e. a polynucleotide sequence encoding at least one amiRNA directed against MAPT) that is under the control of a promoter with which it is spatially connected. A promoter can be positioned 5′ (upstream) or 3′ (downstream) of a polynucleotide sequence under its control. A promoter can be positioned 5′(upstream) of a polynucleotide sequence under its control. The distance between a promoter and a polynucleotide sequence under its control can be approximately the same as the distance between that promoter and the polynucleotide sequence it controls in the gene from which the promoter is derived. Variation in the distance between a promoter and a polynucleotide sequence can be accommodated without loss of promoter function.

In some aspects, a promoter sequence can comprise, consist essentially of, or consist of a JeT promoter sequence. A JeT promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 25, or complement thereof.

In some aspects, a promoter sequence can comprise, consist essentially of, or consist of a MeP229 promoter sequence. A meP229 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 26, or a complement thereof.

In some aspects, a promoter sequence can comprise, consist essentially of, or consist of a MeP426 promoter sequence. A meP426 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 27, or a complement thereof.

In some aspects, a promoter sequence can comprise, consist essentially of, or consist of a CBh promoter sequence. A CBh promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 28, or a complement thereof. A CBh promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 29, or a complement thereof.

In some aspects, a promoter sequence can comprise, consist essentially of, or consist of a U6 promoter sequence. A U6 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 30, or a complement thereof. A murine U6 promoter sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 31, or a complement thereof.

In some aspects, bacterial plasmids of the present disclosure can comprise a prokaryotic promoter. A prokaryotic promoter can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 32, or a complement thereof.

In some embodiments, an rAAV vector provided herein comprises two promoters, for example, a murine U6 promoter and a CBh promoter.

polyA Sequences

In some aspects, a polyadenylation (polyA) sequence can comprise any polyA sequence known in the art. Non-limiting examples of polyA sequences include, but are not limited to, an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence. In some embodiments, the polyA sequence is a synthetic polyA sequence.

Thus, a polyA sequence can comprise, consist essentially of, or consist of an MeCP2 polyA sequence, a retinol dehydrogenase 1 (RDH1) polyA sequence, a bovine growth hormone (BGH) polyA sequence, an SV40 polyA sequence, a SPA49 polyA sequence, a sNRP-TK65 polyA sequence, a sNRP polyA sequence, or a TK65 polyA sequence.

In some aspects, a polyA sequence can comprise, consist essentially of, or consist of an SV40pA sequence. In some aspects, an SV40pA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NOs: 33, or a complement thereof.

In some aspects, a polyA sequence can comprise, consist essentially of, or consist of a BGHpA sequence. In some aspects, an BGHpA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 34, or complement thereof. In some aspects, an BGHpA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 35, or complement thereof.

In some aspects, a synthetic polyA sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to the sequence put forth in SEQ ID NO: 36, or complement thereof.

### Termination Sequence

In some embodiments, an rAAV vector provided herein may comprise a termination sequence. The termination sequence defines the end of a transcriptional unit and initiates the process of releasing the newly synthesized RNA from the transcription machinery. A termination sequence may comprise several thymine residues. For example, a T6 termination comprises the sequence TTTTTT (SEQ ID NO: 40. A T7 terminator comprises the sequence TTTTTTT (SEQ ID NO: 41).

### Bacterial Plasmids

In some aspects, the rAAV vectors of the present disclosure can be contained within a bacterial plasmid to allow for propagation of the rAAV vector in vitro. Thus, the present disclosure provides bacterial plasmids comprising any of the rAAV vectors described herein.

A bacterial plasmid, an rAAV vector, or an rAAV viral vector can further comprise an origin of replication sequence. In some aspects, an origin of replication sequence can comprise, consist essentially of, or consist of any origin of replication sequence known in the art. The origin of replication sequence can be a bacterial origin of replication sequence, thereby allowing the rAAV vector comprising said bacterial origin of replication sequence to be produced, propagated and maintained in bacteria, using methods standard in the art. In some embodiments, the origin of replication comprises, consists essentially of, or consists of the sequences set forth in SEQ ID NO: 36.

A bacterial plasmid, an rAAV vector, or an rAAV viral vector can further comprise an antibiotic resistance gene. In some aspects, an antibiotic resistance gene can comprise, consist essentially of, or consist of any antibiotic resistance genes known in the art. Examples of antibiotic resistance genes known in the art include, but are not limited to kanamycin resistance genes, spectinomycin resistance genes, streptomycin resistance genes, ampicillin resistance genes, carbenicillin resistance genes, bleomycin resistance genes, erythromycin resistance genes, polymyxin B resistance genes, tetracycline resistance genes and chloramphenicol resistance genes. In some embodiments, a kanamycin resistance gene comprises, essentially consists of, or consists of the sequence set forth in SEQ ID NO: 38. In some embodiments, the kanamycin resistance gene is operatively linked to a promoter. In some embodiments, the kanamycin resistance gene is operatively linked to an ampicillin resistance gene promoter (AmpR promoter). In some embodiments, the AmpR promoter comprises, essentially consists of, or consists of the sequence set forth in SEQ ID NO: 37.

A bacterial plasmid, an rAAV vector, or an rAAV viral vector can further comprise a prokaryotic promoter.

### AAV Viral Vectors

A “viral vector” is defined as a recombinantly produced virus or viral particle that contains a polynucleotide to be delivered into a host cell, either in vivo, ex vivo or in vitro. Examples of viral vectors include retroviral vectors, AAV vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

An “AAV virion” or “AAV viral particle” or “AAV viral vector” or “rAAV viral vector” or “AAV vector particle” or “AAV particle” refers to a viral particle composed of at least one AAV capsid protein and an encapsidated polynucleotide rAAV vector. Thus, production of an rAAV viral vector necessarily includes production of an rAAV vector, as such a vector is contained within an rAAV vector.

As used herein, the term “viral capsid” or “capsid” refers to the proteinaceous shell or coat of a viral particle. Capsids function to encapsidate, protect, transport, and release into the host cell a viral genome. Capsids are generally comprised of oligomeric structural subunits of protein (“capsid proteins”). As used herein, the term “encapsidated” means enclosed within a viral capsid. The viral capsid of AAV is composed of a mixture of three viral capsid proteins: VP1, VP2, and VP3. The mixture of VP1, VP2 and VP3 contains 60 monomers that are arranged in a T=1 icosahedral symmetry in a ratio of 1:1:10 (VP1: VP2: VP3) or 1:1:20 (VP1: VP2: VP3) as described in Sonntag F et al., (June 2010). “A viral assembly factor promotes AAV2 capsid formation in the nucleolus”. Proceedings of the National Academy of Sciences of the United States of America. 107 (22): 10220-5, and Rabinowitz JE, Samulski RJ (December 2000). “Building a better vector: the manipulation of AAV virions”. Virology. 278 (2): 301-8, each of which is incorporated herein by reference in its entirety.

The present disclosure provides an rAAV viral vector comprising: a) any of the rAAV vectors described herein, or a complement thereof: and b) an AAV capsid protein.

An AAV capsid protein can be any AAV capsid protein known in the art. An AAV capsid protein can be an AAVI capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

### Compositions and Pharmaceutical Compositions

The present disclosure provides compositions comprising any of the isolated polynucleotides, rAAV vectors, and/or rAAV viral vectors described herein. In some aspects, the compositions can be pharmaceutical compositions. Accordingly, the present disclosure provides pharmaceutical compositions comprising any of the isolated polynucleotides, rAAV vectors, and/or rAAV viral vectors described herein and optionally a pharmaceutically acceptable carrier.

The pharmaceutical composition, as described herein, may be formulated by any methods known or developed in the art of pharmacology, which include but are not limited to contacting the active ingredients (e.g., viral particles or recombinant vectors) with an excipient and/or additive and/or other accessory ingredient, dividing or packaging the product to a dose unit. The viral particles of this disclosure may be formulated with desirable features, e.g., increased stability, increased cell transfection, sustained or delayed release, biodistributions or tropisms, modulated or enhanced translation of encoded protein in vivo, and the release profile of encoded protein in vivo.

As such, the pharmaceutical composition may further comprise saline, lipidoids, liposomes, lipid nanoparticles, polymers, lipoplexes, core-shell nanoparticles, peptides, proteins, cells transfected with viral vectors (e.g., for transplantation into a subject), nanoparticle mimics or combinations thereof. In some aspects, the pharmaceutical composition is formulated as a nanoparticle. In some aspects, the nanoparticle is a self-assembled nucleic acid nanoparticle.

A pharmaceutical composition in accordance with the present disclosure may be prepared, packaged, and/or sold in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. The formulations of the invention can include one or more excipients and/or additives, each in an amount that together increases the stability of the viral vector. increases cell transfection or transduction by the viral vector, increases the expression of viral vector encoded protein, and/or alters the release profile of viral vector encoded proteins. In some aspects, the pharmaceutical composition comprises an excipient and/or additive. Non limiting examples of excipients and/or additives include solvents, dispersion media, diluents, or other liquid vehicles, dispersion or suspension aids, surface active agents, isotonic agents, thickening or emulsifying agents, preservatives, or combination thereof.

In some aspects, the pharmaceutical composition comprises a cryoprotectant. The term “cryoprotectant” refers to an agent capable of reducing or eliminating damage to a substance during freezing. Non-limiting examples of cryoprotectants include sucrose, trehalose, lactose, glycerol, dextrose, raffinose and/or mannitol.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

In some aspects, a pharmaceutical composition of the present disclosure can comprise phosphate-buffered saline, D-sorbitol, sodium chloride, pluronic F-68 or any combination thereof.

In some aspects, a pharmaceutical composition can comprise sodium chloride, wherein the sodium chloride is present at a concentration of about 100 mM to about 500 mM, or about 200 mM to about 400 mM, or about 300 mM to about 400 mM. In some aspects, the sodium chloride can be present at a concentration of about 350 mM.

In some aspects, a pharmaceutical composition can comprise D-sorbitol, wherein the D-sorbitol is present at a concentration of about 1% to about 10%, or about 2.5% to about 7.5%. In some aspects, the D-sorbitol can be present at a concentration of about 5%.

In some aspects, a pharmaceutical composition can comprise pluronic F-68, wherein the pluronic F-68 is present at a concentration of about 0.00001% to about 0.01%, or about 0.0005% to about 0.005%. In some aspects, the pluronic F-68 can be present at a concentration of about 0.001%.

Thus, the present disclosure provides a pharmaceutical composition comprising an rAAV vector and/or rAAV viral vector of the present disclosure in a phosphate-buffered saline solution, wherein the pharmaceutical composition further comprises sodium chloride at a concentration of 350 mM, D-sorbitol at a concentration of 5% and pluronic F-68 at a concentration of 0.001%.

Thus, the present disclosure provides a pharmaceutical composition comprising an rAAV vector and/or rAAV viral vector of the present disclosure, wherein the pharmaceutical composition further comprises sodium chloride at a concentration of 350 mM, D-sorbitol at a concentration of 5% and pluronic F-68 at a concentration of 0.001%.

Thus, the present disclosure provides a pharmaceutical composition comprising an rAAV vector and/or rAAV viral vector of the present disclosure in a phosphate-buffered saline solution, wherein the pharmaceutical composition further comprises sodium chloride at a concentration of 350 mM, D-sorbitol at a concentration of 5%.

Thus, the present disclosure provides a pharmaceutical composition comprising an rAAV vector and/or rAAV viral vector of the present disclosure, wherein the pharmaceutical composition further comprises sodium chloride at a concentration of 350 mM, D-sorbitol at a concentration of 5%.

### Methods of Using the Compositions of the Disclosure

The present disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

This disclosure provides methods of preventing or treating a disorder, comprising, consisting essentially of, or consisting of administering to a subject a therapeutically effective amount of any one of the isolated nucleic acid molecules, rAAV vectors, rAAV viral vectors, compositions and/or pharmaceutical compositions disclosed herein.

In one aspect, provided herein is a method of treating a tauopathy in a subject, the method comprising administering to the subject and effective amount of an isolated nucleic acid, an rAAV vector, an rAAV viral vector, or a composition described herein.

In some embodiments, the subject suffers from a disorder that is associated a mutation in the MAPT gene. In some embodiments, a subject treated in accordance with the methods described herein has one or more mutations in the MAPT gene. Mutations in MAPT that have an effect on Tau function are known in the art, see. e.g., Strang et al., Lab Invest. 2019 Jul; 99(7): 912-928. In some embodiments, the mutation is selected from the group consisting of R5H, R5L, G55R, K257T, 1260V, L266V, G272V, N279K, A280K, S285R, K298E, P301L, P301S, P301T, G303V, S305I, S305N, K317M, K317N, S320F, P332S, G335V, Q336H, Q336R, V337M, S352L, S356T, P364S, K369I, E372G, G389R (G→A), G389R (G→C), and N410H.

In some embodiments, the disease is AD. In some embodiments, the disease is FNTD-17.

Accordingly, the present disclosure provides methods of preventing or treating a tauopathy in a subject, comprising, consisting essentially of, or consisting of administering to the subject a therapeutically effective amount of any one of the isolated nucleic acid molecules, rAAV vectors, rAAV viral vectors, compositions and/or pharmaceutical compositions disclosed herein. The present disclosure provides the use of any one of the isolated nucleic acid molecules, rAAV vectors, rAAV viral vectors, compositions and/or pharmaceutical compositions disclosed herein for the manufacture of a medicament for the treatment or prevention of a tauopathy. The present disclosure provides any one of the isolated nucleic acid molecules, rAAV vectors, rAAV viral vectors, compositions and/or pharmaceutical compositions disclosed herein for use in treating or preventing a tauopathy. In some embodiments, the tauopathy is AD. In some embodiments, the disease is FNTD-17.

The methods of treatment and prevention disclosed herein may be combined with appropriate diagnostic techniques to identify and select patients for the therapy or prevention.

The disclosure provides methods of decreasing the level of one or more proteins in a host cell, comprising contacting the host cell with any one of the rAAV viral vectors disclosed herein, wherein the rAAV viral vectors comprises any one of the rAAV vectors disclosed herein. In some aspects, the host cell is in vitro, in vivo, or ex vivo. In some aspects, the host cell is derived from a subject, e.g., a subject suffering from a tauopathy.

In some aspects, administration of an isolated nucleic acid, an rAAV vector or an rAAV viral vector herein results in a decrease in Tau protein expression in the host cell. The level of Tau may be decreased to level of about 1×10⁻⁷ ng, about 3×10⁻⁷ ng, about 5×10⁻⁷ ng, about 7×10⁻⁷ ng, about 9 ×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

In some embodiments, the levels of Tau protein may be decreased by at least about 5%, at least about 10%, at least about 15%, at least about 20%, at least about 25%, at least about 30%, at least about 35%, at least about 40%, at least about 45%, at least about 50%, at least about 55%, at least about 65%, at least about 70%, at least about 75%, at least about 80%, at least about 90%, at least about 95%, or about 100%.

In some embodiments, the levels of Tau protein may be decreased at least about 1.25 fold, at least bout 1.5 fold, at least about 1.25 fold, at least about 2 fold, at least about 2.25 fold, at least about 2.5 fold, at least about 2.75 fold, at least about 3 fold, at least about 4 fold, at least about 5 fold, at least about 6 fold, at least about 7 fold, at least about 8 fold, at least about 9 fold, or at least about 10 fold.

The disclosure provides methods of decreasing the level of an RNA molecule in a host cell, comprising contacting the host cell with any one of the rAAV viral vectors disclosed herein, wherein the rAAV viral vectors comprises any one of the rAAV vectors disclosed herein. In some aspects the RNA molecule is an mRNA molecule or a non-coding RNA molecule. In some aspects, the host cell is in vitro, in vivo, or ex vivo. In some aspects, the host cell is derived from a subject, e.g., a subject suffering from a tauopathy. In some embodiments, the host cell is a cell from the brain stem, the cerebellum, or the cortex of a subject suffering from a tauopathy.

In some aspects, administration of an isolated nucleic acid, an rAAV vector or an rAAV viral vector herein results in a decrease in MAPT mRNA in the host cell. The level of the mRNA molecule may be decreased to level of about 1×10⁻⁷ ng, about 3×10⁻⁷ ng, about 5×10⁻⁷ ng, about 7×10⁻⁷ ng, about 9×10⁻⁷ ng, about 1×10⁻⁶ ng, about 2×10⁻⁶ ng, about 3×10⁻⁶ ng, about 4×10⁻⁶ ng, about 6×10⁻⁶ ng, about 7×10⁻⁶ ng, about 8×10⁻⁶ ng, about 9×10⁻⁶ ng, about 10×10⁻⁶ ng, about 12×10⁻⁶ ng, about 14×10⁻⁶ ng, about 16×10⁻⁶ ng, about 18×10⁻⁶ ng, about 20×10⁻⁶ ng, about 25×10⁻⁶ ng, about 30×10⁻⁶ ng, about 35×10⁻⁶ ng, about 40×10⁻⁶ ng, about 45×10⁻⁶ ng, about 50×10⁻⁶ ng, about 55×10⁻⁶ ng, about 60×10⁻⁶ ng, about 65×10⁻⁶ ng, about 70×10⁻⁶ ng, about 75×10⁻⁶ ng, about 80×10⁻⁶ ng, about 85×10⁻⁶ ng, about 90×10⁻⁶ ng, about 95×10⁻⁶ ng, about 10×10⁻⁵ ng, about 20×10⁻⁵ ng, about 30×10⁻⁵ ng, about 40×10⁻⁵ ng, about 50×10⁻⁵ ng, about 60×10⁻⁵ ng, about 70×10⁻⁵ ng, about 80×10⁻⁵ ng, or about 90×10⁻⁵ ng in the host cell.

In some embodiments, the level of MAPT mRNA is decreased by at least about 5%, at least about 10%, at least about 15%, at least about 20%, at least about 25%, at least about 30%, at least about 35%, at least about 40%, at least about 45%, at least about 50%, at least about 55%, at least about 65%, at least about 70%, at least about 75%, at least about 80%, at least about 90%, at least about 95%, or about 100%.

In some embodiments, the level of MAPT mRNA is decreased at least about 1.25 fold, at least about 1.5 fold, at least about 1.25 fold, at least about 2 fold, at least about 2.25 fold, at least about 2.5 fold, at least about 2.75 fold, at least about 3 fold, at least about 4 fold, at least about 5 fold, at least about 6 fold, at least about 7 fold, at least about 8 fold, at least about 9 fold, or at least about 10 fold.

The disclosure provides methods of introducing a polynucleotide sequence of interest (e.g. a polynucleotide sequence encoding at least one amiRNA directed against MAPT) to a cell in a subject comprising contacting the cell with an effective amount of any one of the rAAV viral vectors disclosed herein, wherein the rAAV viral vectors contain any one of the rAAV vectors disclosed herein, comprising the polynucleotide sequence of interest.

In some aspects of the methods of the present disclosure, a subject can also be administered a prophylactic immunosuppressant treatment regimen in addition to being administered an rAAV vector or rAAV viral vector of the present disclosure. In some aspects, an immunosuppressant treatment regimen can comprise administering at least one immunosuppressive therapeutic. Non limiting examples of immunosuppressive therapeutics include, but are not limited to, Sirolimus (rapamycin), acetaminophen, diphenhydramine, IV methylprednisolone, prednisone, or any combination thereof. An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

A “subject” of diagnosis or treatment is a cell or an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In some aspects, the subject is a human. The terms “subject” and “patient” are used interchangeably herein.

As used herein, “treating” or “treatment” of a disease in a subject refers to (1) delaying the symptoms or disease from occurring in a subject that is predisposed or does not yet display symptoms of the disease: (2) inhibiting the disease or arresting its development: or (3) ameliorating or causing regression of the disease or the symptoms of the disease. The delaying, inhibiting and ameliorating may be compared to another subject (e.g., a subject suffering from the same tauopathy (e.g., AD or FTDP-17)) not being administered a composition, an isolated nucleic acid molecule, an rAAV vector, or rAAV viral vector described herein). The inhibiting or ameliorating may be compared to another subject (e.g., a subject suffering from a tauopathy (e.g., AD or FTDP-17) not being administered a composition, an isolated nucleic acid molecule, an rAAV vector, or rAAV viral vector described herein) or to the subject being treated prior to the first administration of the composition, isolated nucleic acid molecule, rAAV vector, or rAAV viral vector described herein.

As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

In some embodiments, the symptom being ameliorated by administration of a composition, an isolated nucleic acid molecule, an rAAV vector, or rAAV viral vector described herein is the accumulation of Tau, in particular, hyperphosphorylated, filamentous Tau, in the brain. Tau protein may be detected using any suitable method known in the art or described herein, including, for example, immunohistochemistry.

In some embodiments, the symptom being ameliorated by administration of a composition, an isolated nucleic acid molecule, an rAAV vector, or rAAV viral vector described herein is a dementia.

As used herein the term “effective amount” intends to mean a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of gene therapy, the effective amount can be the amount sufficient to result in normal or improved function of a gene that is deficient in a subject. In some embodiments, the effective amount of an isolated polynucleotide, an rAAV vector, an rAAV viral vector or a composition provided herein is the amount sufficient to result in expression of an amiRNA directed against MAPT in the subject, e.g., in the brain stem, the cortex, and/or the cerebellum of the subject. In some embodiments, the effective amount of an isolated polynucleotide, an rAAV vector, an rAAV viral vector or a composition provided herein is the amount sufficient to result in expression of an amiRNA directed against MAPT in the subject such that the expression of Tau is upregulated. In some embodiments, the expression of Tau is upregulated in the brain stem, the cortex, and/or the cerebellum. The skilled artisan will be able to determine appropriate amounts depending on these and other factors.

In some aspects, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administer” or “administration” intends to mean delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. It is noted that dosage may be impacted by the route of administration. Suitable dosage formulations and methods of administering the agents are known in the art. Non-limiting examples of such suitable dosages may be as low as 10⁹ vector genomes to as much as 10¹⁷ vector genomes (or “viral particles”) per administration.

In some aspects, the amounts of viral particles in a composition, pharmaceutical composition, or the amount of viral particles administered to a patient can calculated based on the percentage of viral particles that are predicted to contain viral genomes.

In some aspects, rAAV viral vectors of the present disclosure can be introduced to the subject intravenously, intrathecally, intracerebrally, intra-cisternal magna (ICM), intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally: such introduction may also be intra-arterial, intracardiac, subventricular, epidural, intracerebral, intracerebroventricular, sub-retinal, intravitreal, intraarticular, intraperitoneal, intrauterine, intranerve or any combination thereof. In some aspects, the viral particles are delivered to a desired target tissue, e.g., to the lung, eye, or CNS, as non-limiting examples. In some aspects, delivery of viral particles is systemic. The intracisternal route of administration involves administration of a drug directly into the cerebrospinal fluid of the brain ventricles. It could be performed by direct injection into the cisterna magna or via a permanently positioned tube. In some aspects, the rAAV viral vectors of the present disclosure are administered intrathecally. In some aspects, the rAAV viral vectors of the present disclosure are administered intra-cisterna magna.

Administration of the rAAV vectors, rAAV viral vectors, compositions or pharmaceutical compositions of this disclosure can be effected in one dose, continuously or intermittently throughout the course of treatment. In some aspects, the rAAV vectors, rAAV viral vectors, compositions, or pharmaceutical compositions of this disclosure are parenterally administered by injection, infusion, or implantation.

In some aspects, the rAAV viral vectors of this disclosure show enhanced tropism for brain and cervical spine. In some aspects, the rAAV viral vectors of the disclosure can cross the blood-brain-barrier (BBB).

### Methods of Manufacture

A variety of approaches may be used to produce or rAAV viral vectors of the present disclosure. In some aspects, packaging is achieved by using a helper virus or helper plasmid and a cell line. The helper virus or helper plasmid contains elements and sequences that facilitate viral vector production. In another aspect, the helper plasmid is stably incorporated into the genome of a packaging cell line, such that the packaging cell line does not require additional transfection with a helper plasmid.

In some aspects, the cell is a packaging or helper cell line. In some aspects, the helper cell line is eukaryotic cell: for example, an HEK 293 cell or 293T cell. In some aspects, the helper cell is a yeast cell or an insect cell.

In some aspects, the cell comprises a nucleic acid encoding a tetracycline activator protein; and a promoter that regulates expression of the tetracycline activator protein. In some aspects, the promoter that regulates expression of the tetracycline activator protein is a constitutive promoter. In some aspects, the promoter is a phosphoglycerate kinase promoter (PGK) or a CMV promoter.

A helper plasmid may comprise, for example, at least one viral helper DNA sequence derived from a replication-incompetent viral genome encoding in trans all virion proteins required to package a replication incompetent AAV, and for producing virion proteins capable of packaging the replication-incompetent AAV at high titer, without the production of replication-competent AAV.

Helper plasmids for packaging AAV are known in the art, sec, e.g., U.S. Patent Pub. No. 2004/0235174 A1, incorporated herein by reference. As stated therein, an AAV helper plasmid may contain as helper virus DNA sequences, by way of non-limiting example, the Ad5 genes E2A, E4 and VA, controlled by their respective original promoters or by heterologous promoters. AAV helper plasmids may additionally contain an expression cassette for the expression of a marker protein such as a fluorescent protein to permit the simple detection of transfection of a desired target cell.

The disclosure provides methods of producing rAAV viral vectors comprising transfecting a packaging cell line with any one of the AAV helper plasmids disclosed herein: and any one of the rAAV vectors disclosed herein. In some aspects, the AAV helper plasmid and rAAV vector are co-transfected into the packaging cell line. In some aspects, the cell line is a mammalian cell line, for example, human embryonic kidney (HEK) 293 cell line. The disclosure provides cells comprising any one of the rAAV vectors and/or rAAV viral vectors disclosed herein.

As used herein, the term “helper” in reference to a virus or plasmid refers to a virus or plasmid used to provide the additional components necessary for replication and packaging of any one of the rAAV vectors disclosed herein. The components encoded by a helper virus may include any genes required for virion assembly, encapsidation, genome replication, and/or packaging. For example, the helper virus or plasmid may encode necessary enzymes for the replication of the viral genome. Non-limiting examples of helper viruses and plasmids suitable for use with AAV constructs include pHELP (plasmid), adenovirus (virus), or herpesvirus (virus). In some aspects, the pHELP plasmid may be the pHELPK plasmid, wherein the ampicillin expression cassette is exchanged with a kanamycin expression cassette.

As used herein, a packaging cell (or a helper cell) is a cell used to produce viral vectors. Producing recombinant AAV viral vectors (rAAV viral vectors) requires Rep and Cap proteins provided in trans as well as gene sequences from Adenovirus that help AAV replicate. In some aspects, Packaging/helper cells contain a plasmid is stably incorporated into the genome of the cell. In other aspects, the packaging cell may be transiently transfected. Typically, a packaging cell is a eukaryotic cell, such as a mammalian cell or an insect cell.

### Kits

The isolated polynucleotides, rAAV vectors, rAAV viral vectors, compositions, and/or pharmaceutical compositions described herein may be assembled into pharmaceutical or diagnostic or research kits to facilitate their use in therapeutic, diagnostic, or research applications. In some aspects, the kits of the present disclosure include any one of the isolated polynucleotides, rAAV vectors, rAAV viral vectors, compositions, pharmaceutical compositions, host cells, isolated tissues, as described herein.

In some aspects, a kit further comprises instructions for use. Specifically, such kits may include one or more agents described herein, along with instructions describing the intended application and the proper use of these agents. In some aspects, the kit may include instructions for mixing one or more components of the kit and/or isolating and mixing a sample and applying to a subject. In some aspects, agents in a kit are in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. In some aspects, the compositions may be provided in a preservation solution (e.g., cryopreservation solution). Non-limiting examples of preservation solutions include DMSO, paraformaldehyde, and CryoStor R® (Stem Cell Technologies, Vancouver, Canada). In some aspects, the preservation solution contains an amount of metalloprotease inhibitors.

In some aspects, the kit contains any one or more of the components described herein in one or more containers. Thus, in some aspects, the kit may include a container housing agents described herein. The agents may be in the form of a liquid, gel or solid (powder). The agents may be prepared sterilely, packaged in a syringe and shipped refrigerated. Alternatively, they may be housed in a vial or other container for storage. A second container may have other agents prepared sterilely.

Alternatively, the kit may include the active agents premixed and shipped in a syringe, vial, tube, or other container. The kit may have one or more or all of the components required to administer the agents to a subject, such as a syringe, topical application devices, or IV needle tubing and bag.

### Further Definitions

Unless the context indicates otherwise, it is specifically intended that the various features of the invention described herein can be used in any combination. Moreover, the disclosure also contemplates that, in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified aspects, embodiments, features, and terms intend to include both the recited aspect, embodiment, feature, or term and biological equivalents thereof.

The practice of the present technology will employ, unless otherwise indicated, conventional techniques of organic chemistry, pharmacology, immunology, molecular biology, microbiology, cell biology and recombinant DNA, which are within the skill of the art. Sec, e.g., Sambrook, Fritsch and Maniatis, Molecular Cloning: A Laboratory Manual, 2nd edition (1989): Current Protocols In Molecular Biology (F. M. Ausubel, et al. eds., (1987)); the series Methods in Enzymology (Academic Press, Inc.): PCR 2: A Practical Approach (M.J. MacPherson, B.D. Hames and G.R. Taylor eds. (1995)), Harlow and Lane, eds. (1988) Antibodies, a Laboratory Manual, and Animal Cell Culture (RI. Freshney, cd. (1987)).

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure. In each instance herein any of the terms “comprising.” “consisting essentially of,” and “consisting of” can be replaced with either of the other two terms, while retaining their ordinary meanings.

All numerical designations, e.g., pH, temperature, time, concentration, and molecular weight, including ranges, are approximations which are varied (+) or (−) by increments of 1.0 or 0.1, as appropriate, or, alternatively, by a variation of +/−15%, 10%, 5%, 2%. It is to be understood, although not always explicitly stated, that all numerical designations are preceded by the term “about”. It also is to be understood, although not always explicitly stated, that the reagents described herein are merely exemplary and that equivalents of such are known in the art. The term “about,” as used herein when referring to a measurable value such as an amount or concentration and the like, is meant to encompass variations of 20%, 10%, 5%, 1%, 0.5%, or even 0.1% of the specified amount.

The terms “acceptable,” “effective,” or “sufficient” when used to describe the selection of any components, ranges, dose forms, etc. disclosed herein intend that said component, range, dose form, etc. is suitable for the disclosed purpose.

Also, as used herein, “and/or” refers to and encompasses any and all possible combinations of one or more of the associated listed items, as well as the lack of combinations when interpreted in the alternative (“or”).

Unless specifically recited, the term “host cell” includes a eukaryotic host cell, including, for example, fungal cells, yeast cells, higher plant cells, insect cells and mammalian cells. Non-limiting examples of eukaryotic host cells include simian, bovine, porcine, murine, rat, avian, reptilian and human, e.g., HEK293 cells and 293T cells.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically or biochemically modified, non-natural, or derivatized nucleotide bases.

A “gene” refers to a polynucleotide containing at least one open reading frame (ORF) that is capable of encoding a particular polypeptide or protein. A “gene product” or, alternatively, a “gene expression product” refers to the amino acid sequence (e.g., peptide or polypeptide) generated when a gene is transcribed and translated.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a cukaryotic cell.

“Under transcriptional control” is a term well understood in the art and indicates that transcription of a polynucleotide sequence, usually a DNA sequence, depends on its being operatively linked to an element that contributes to the initiation of, or promotes, transcription. “Operatively linked” intends that the polynucleotides are arranged in a manner that allows them to function in a cell. In one aspect, promoters can be operatively linked to the downstream sequences.

The term “encode” as it is applied to polynucleotide sequences and/or nucleic acid sequences refers to a polynucleotide sequences and/or nucleic acid sequences which are said to “encode” an RNA molecule (e.g. an shRNA molecule) if, in their native state, the of the polynucleotide sequence and/or nucleic acid sequence corresponds the sequence of the shRNA molecule that is biologically active. The antisense strand is the complement of such a polynucleotide sequence and/or nucleic acid sequence, and the encoding sequence can be deduced therefrom.

The terms “equivalent” or “biological equivalent” are used interchangeably when referring to a particular molecule, biological material, or cellular material and intend those having minimal homology while still maintaining desired structure or functionality. Non-limiting examples of equivalent polypeptides include a polypeptide having at least about 60%, at least about 65%, at least about 70%, at least about 75%, at least about 80%, at least about 85%, at least about 90%, at least about 95% identity or at least about 99% identity to a reference polypeptide (for instance, a wild-type polypeptide): or a polypeptide which is encoded by a polynucleotide having at least about 70%, at least about 75%, at least about 80%, at least about 85%, at least about 90%, at least about 95% identity, at least about 97% sequence identity or at least about 99% sequence identity to the reference polynucleotide (for instance, a wild-type polynucleotide).

“Homology” or “identity” or “similarity” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences. “Unrelated” or “non-homologous” sequences share less than 40% identity, less than 25% identity, with one of the sequences of the present disclosure. Alignment and percent sequence identity may be determined for the nucleic acid or amino acid sequences provided herein by importing said nucleic acid or amino acid sequences into and using ClustalW (available at https://genome.jp/tools-bin/clustalw/). For example, the ClustalW parameters used for performing the protein sequence alignments found herein were generated using the Gonnet (for protein) weight matrix. In some aspects, the ClustalW parameters used for performing nucleic acid sequence alignments using the nucleic acid sequences found herein are generated using the ClustalW (for DNA) weight matrix.

A polynucleotide disclosed herein can be delivered to a cell or tissue using a gene delivery vehicle. “Gene delivery.” “gene transfer,” “transducing.” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is a DNA molecule that is typically separate from and capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or, alternatively, the proteins produced may act as toxins under similar circumstances. It is known in the art that while plasmid vectors often exist as extrachromosomal circular DNA molecules, plasmid vectors may also be designed to be stably integrated into a host chromosome either randomly or in a targeted manner, and such integration may be accomplished using either a circular plasmid or a plasmid that has been linearized prior to introduction into the host cell.

“Plasmids” used in genetic engineering are called “plasmid vectors”. Many plasmids are commercially available for such uses. The gene to be replicated is inserted into copies of a plasmid containing genes that make cells resistant to particular antibiotics, and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria or eukaryotic cells containing a plasmid harboring the gene of interest, which can be induced to produce large amounts of proteins from the inserted gene.

In aspects where gene transfer is mediated by a DNA viral vector, such as an adenovirus (Ad) or adeno-associated virus (AAV), a vector construct refers to the polynucleotide comprising, consisting essentially of, or consisting of the viral genome or part thereof, and a transgene/exogenous polynucleotide sequence.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected), or a group of tissues making up an organ or part or region of the body of an organism. The tissue may comprise, consist essentially of, or consist of a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

All references cited herein are incorporated herein by reference in their entirety.

### EXAMPLES

The examples described in this section is provided for illustration only and is in no way intended to limit the invention.

### Example 1: Screen for miRNAs Targeting Human or Mouse MAPT

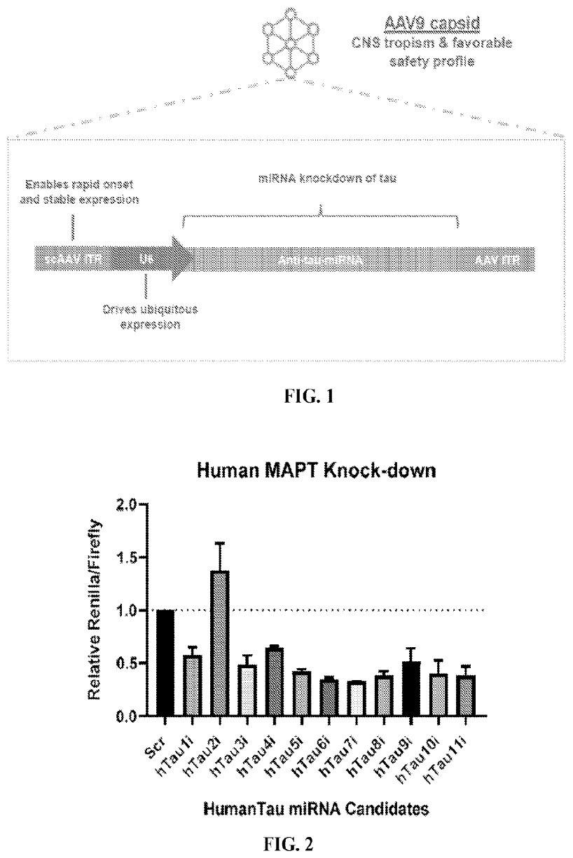

As a novel therapeutic approach for Tauopathies, AAV delivery of artificial miRNA shuttles was used to target tau mRNA encoded by the MAPT gene. The antisense guide-strands of the miRNA contains perfect complementarity with tau mRNA across a 22 nt stretch of sequence, thereby directing the transcript toward an RNAi degradation pathway. FIG. 1 shows a schematic of the targeting of Tau using miRNA. Evidence supports that miRNA shuttles are more predictably processed, efficient, and safer than shRNAs (Boudreau et al., Mol Ther 17, 169-175, doi: 10.1038/mt.2008.231 (2009); Grimm et al. Nature 441, 537-541, doi: 10.1038/nature04791 (2006)) and they can be expressed in vivo from DNA-based delivery systems, such as viral vectors.

A panel of mir-30-based artificial miRNA shuttles were designed to be specific to sequences shared in all tau brain isoforms. Reference sequences for tau- or mouse-specific miRNA shuttles were generated using an AppleScript program that uses published constraints to predict miRNA shuttle sequences based on the target gene that is inputted by the user. The mature guide strand were designed to specifically target only human tau (hTaui), mouse tau (mTaui) or to target both human and mouse tau (hmTaui) mRNA for degradation. From the list generated by the program, potential candidates were selected based on screening for species specificity and potential off-target genes. Using the published protocol from Boudreau et al. (RNA Interference Techniques Neuromethods (ed S. Q. Harper) Ch. 2, 173 (Humana Press, 2011), the miRNA shuttle sequences were synthesized and cloned into the U6T6 plasmid.

To screen for target engagement, 10-20 candidates from each species-specific library were cloned into the PsiCheck2 plasmid and a dual-luciferase reporter assay (DLRA) was performed following standard practices. The shuttle DNA sequences encoding the tested constructs are set forth in SEQ ID NOs: 41-89, the corresponding RNA sequences are set forth in SEQ ID NOs: 90-138, and the amiRNA sequences directed against MAPT are set forth in SEQ ID NOs: 139-187 as shown in Table 2.

TABLE 2
Tested Constructs

| miRNA | Target | miRNA Shuttle | Full length miRNA | amiRNA |
|---|---|---|---|---|
| Shuttle | Species | DNA (5′-3′) | Sequence (5′-3′) | Sequence (5′-3″) |
| hTau1i | Human | SEQ ID NO: 41 | SEQ ID NO: 90 | SEQ ID NO: 139 |
| hTau2i | Human | SEQ ID NO: 42 | SEQ ID NO: 91 | SEQ ID NO: 140 |
| hTau3i | Human | SEQ ID NO: 43 | SEQ ID NO: 92 | SEQ ID NO: 141 |
| hTau4i | Human | SEQ ID NO: 44 | SEQ ID NO: 93 | SEQ ID NO: 142 |
| hTau5i | Human | SEQ ID NO: 45 | SEQ ID NO: 94 | SEQ ID NO: 143 |
| hTau6i | Human | SEQ ID NO: 46 | SEQ ID NO: 95 | SEQ ID NO: 144 |
| hTau7i | Human | SEQ ID NO: 47 | SEQ ID NO: 96 | SEQ ID NO: 145 |
| hTau8i | Human | SEQ ID NO: 48 | SEQ ID NO: 97 | SEQ ID NO: 146 |
| hTau9i | Human | SEQ ID NO: 49 | SEQ ID NO: 98 | SEQ ID NO: 147 |
| hTau10i | Human | SEQ ID NO: 50 | SEQ ID NO: 99 | SEQ ID NO: 148 |
| hTau11i | Human | SEQ ID NO: 51 | SEQ ID NO: 100 | SEQ ID NO: 149 |
| mTau1i | Mouse | SEQ ID NO: 52 | SEQ ID NO: 101 | SEQ ID NO: 150 |
| mTau2i | Mouse | SEQ ID NO: 53 | SEQ ID NO: 102 | SEQ ID NO: 151 |
| mTau3i | Mouse | SEQ ID NO: 54 | SEQ ID NO: 103 | SEQ ID NO: 152 |
| mTau4i | Mouse | SEQ ID NO: 55 | SEQ ID NO: 104 | SEQ ID NO: 153 |
| mTau5i | Mouse | SEQ ID NO: 56 | SEQ ID NO: 105 | SEQ ID NO: 154 |
| mTau6i | Mouse | SEQ ID NO: 57 | SEQ ID NO: 106 | SEQ ID NO: 155 |
| mTau7i | Mouse | SEQ ID NO: 58 | SEQ ID NO: 107 | SEQ ID NO: 156 |
| mTau8i | Mouse | SEQ ID NO: 59 | SEQ ID NO: 108 | SEQ ID NO: 157 |
| mTau9i | Mouse | SEQ ID NO: 60 | SEQ ID NO: 109 | SEQ ID NO: 158 |
| mTau10i | Mouse | SEQ ID NO: 61 | SEQ ID NO: 110 | SEQ ID NO: 159 |
| mTau11i | Mouse | SEQ ID NO: 62 | SEQ ID NO: 111 | SEQ ID NO: 160 |
| mTau12i | Mouse | SEQ ID NO: 63 | (SEQ ID NO: 112 | SEQ ID NO: 161 |
| mTau13i | Mouse | SEQ ID NO: 64 | SEQ ID NO: 113 | SEQ ID NO: 162 |
| mTau14i | Mouse | SEQ ID NO: 65 | SEQ ID NO: 114 | SEQ ID NO: 163 |
| mTau15i | Mouse | SEQ ID NO: 66 | SEQ ID NO: 115 | SEQ ID NO: 164 |
| mTau16i | Mouse | SEQ ID NO: 67 | SEQ ID NO: 116 | SEQ ID NO: 165 |
| mTau17i | Mouse | SEQ ID NO: 68 | SEQ ID NO: 117 | SEQ ID NO: 166 |
| mTau18i | Mouse | SEQ ID NO: 69 | SEQ ID NO: 118 | SEQ ID NO: 167 |
| mTau19i | Mouse | SEQ ID NO: 70 | SEQ ID NO: 119 | SEQ ID NO: 168 |
| hmTau1i | Human and | SEQ ID NO: 71 | SEQ ID NO: 120 | SEQ ID NO: 169 |
| | Mouse | | | |
| hmTau2i | Human and | SEQ ID NO: 72 | SEQ ID NO: 121 | SEQ ID NO: 170 |
| | Mouse | | | |
| hmTau3i | Human and | SEQ ID NO: 73 | SEQ ID NO: 122 | SEQ ID NO: 171 |
| | Mouse | | | |
| hmTau4i | Human and | SEQ ID NO: 74 | SEQ ID NO: 123 | SEQ ID NO: 172 |
| | Mouse | | | |
| hmTau5i | Human and | SEQ ID NO: 75 | SEQ ID NO: 124 | SEQ ID NO: 173 |
| | Mouse | | | |
| hmTau6i | Human and | SEQ ID NO: 76 | SEQ ID NO: 125 | SEQ ID NO: 174 |
| | Mouse | | | |
| hmTau7i | Human and | SEQ ID NO: 77 | SEQ ID NO: 126 | SEQ ID NO: 175 |
| | Mouse | | | |
| hmTau8i | Human and | SEQ ID NO: 78 | SEQ ID NO: 127 | SEQ ID NO: 176 |
| | Mouse | | | |
| hmTau9i | Human and | SEQ ID NO: 79 | SEQ ID NO: 128 | SEQ ID NO: 177 |
| | Mouse | | | |
| hmTau10i | Human and | SEQ ID NO: 80 | SEQ ID NO: 129 | SEQ ID NO: 178 |
| | Mouse | | | |
| hmTau11i | Human and | SEQ ID NO: 81 | SEQ ID NO: 130 | SEQ ID NO: 179 |
| | Mouse | | | |
| hmTau12i | Human and | SEQ ID NO: 82 | SEQ ID NO: 131 | SEQ ID NO: 180 |
| | Mouse | | | |
| hmTau13i | Human and | SEQ ID NO: 83 | SEQ ID NO: 132 | SEQ ID NO: 181 |
| | Mouse | | | |
| hmTau14i | Human and | SEQ ID NO: 84 | SEQ ID NO: 133 | SEQ ID NO: 182 |
| | Mouse | | | |
| hmTau15i | Human and | SEQ ID NO: 85 | SEQ ID NO: 134 | SEQ ID NO: 183 |
| | Mouse | | | |
| hmTau16i | Human and | SEQ ID NO: 86 | SEQ ID NO: 135 | SEQ ID NO: 184 |
| | Mouse | | | |
| hmTau17i | Human and | SEQ ID NO: 87 | SEQ ID NO: 135 | SEQ ID NO: 185 |
| | Mouse | | | |
| hmTau18i | Human and | SEQ ID NO: 88 | SEQ ID NO: 137 | SEQ ID NO: 186 |
| | Mouse | | | |
| Scramble | — | SEQ ID NO: 89 | SEQ ID NO: 138 | SEQ ID NO: 187 |

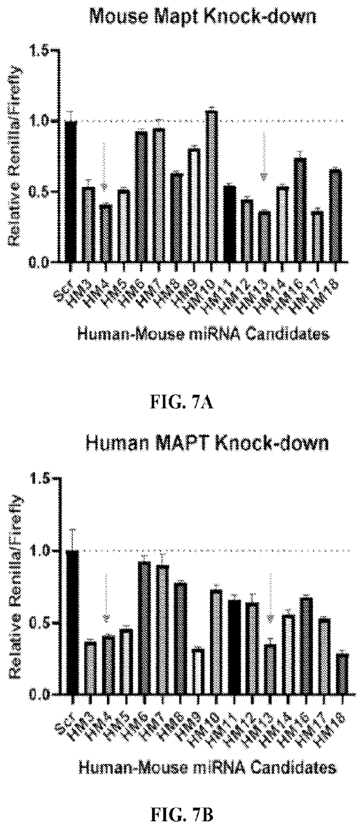

Results of the primary screen of miRNAs targeting human tau are shown in FIG. 2 and results of the primary screen of miRNAs targeting mouse tau are shown in FIG. 5. Results of a primary screen of miRNAs targeting both human and mouse Tau are shown in FIGS. 7A and 7B.

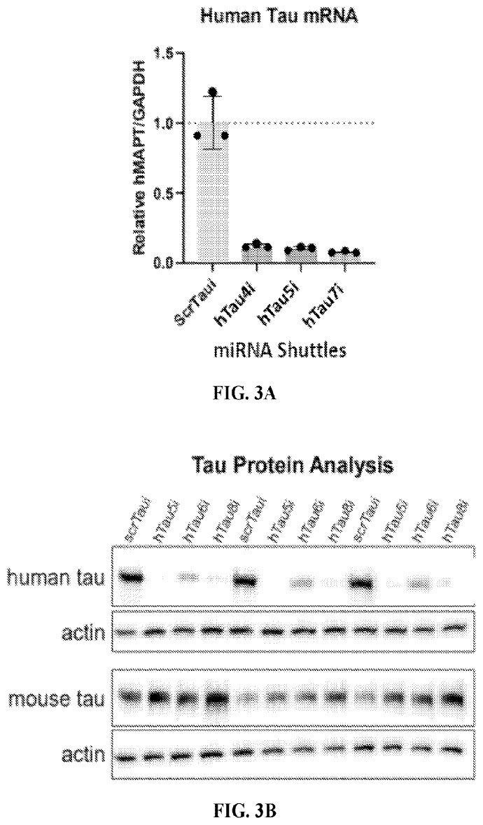

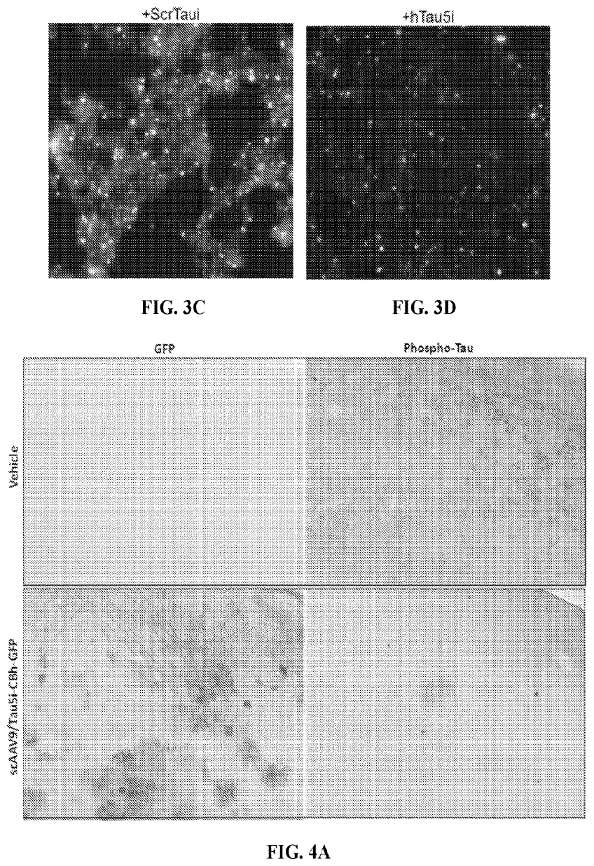

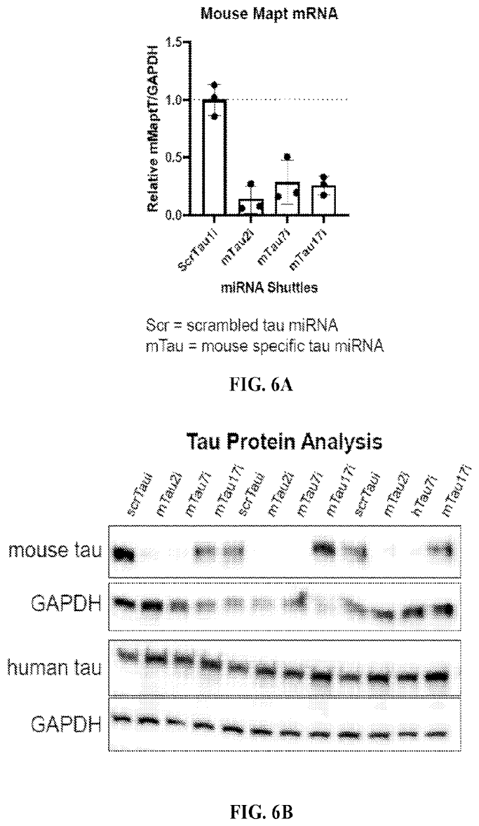

From this primary screen. the top three candidates were then tested in a secondary screen where the miRNA shuttle plasmid was co-transfected with a plasmid expressing the human or mouse cDNA in HEK293T cells. Results of the secondary screen of miRNAs targeting human tau are shown in FIGS. 3A-3D. and results of the secondary screen of miRNAs targeting mouse tau are shown in FIGS. 6A and 6B.

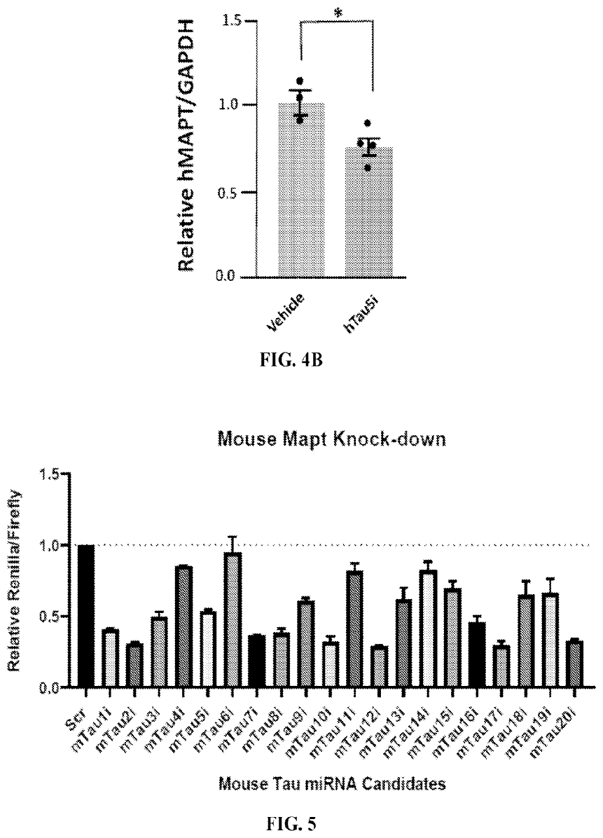

The top candidate from the secondary screen was then cloned into an AAV vector for viral delivery. First generation Tau miRNA shuttle vectors have the hTaui or mTaui shuttles upstream of a GFP reporter transgene within the same cassette. This design allows the expression of GFP protein in transduced cells to allow for assessments of viral biodistribution. FIGS. 4A and 4B show vector expression and target engagement of AAV9/hTaui-GFP in P301S tau mice. In second generation Tau miRNA shuttles. the GFP coding sequence has been dropped to allow for use in humans.

### Example 2: Study to Evaluate the Efficacy of AAV9/hTau5i when Administered Intra-Cisterna Magna in 3 Month-Old P301S Tau Mice

This study was designed to assess the therapeutic benefit of AAV9/hTau5i in the PS 19 mouse model of tauopathy early in the disease course. The sequence of AAV9/hTauSi is set forth in SEQ ID NO: 14. PS19 mice overexpress human P301S mutant tau at 7-fold greater levels than endogenous mouse tau. This model develops neuronal loss and brain atrophy by 8 months. primarily in the hippocampus and spreading to other brain regions such as the neocortex and entorhinal cortex. These mice develop widespread neurofibrillary tangle-like inclusions in the neocortex. hippocampus. brainstem and spinal cord (Yoshiyama et al., Neuron. 2007 Feb 1:53(3):337-51). Prior to the appearance of overt tau pathology by histological methods. brains of these mice display seeding activity where tau can propagate from one cell to another and along synaptically connected brain networks (Holmes et al., Proc Natl Acad Sci U S A. 2013 Aug 13:110(33): E3138-47). It has been shown that tau aggregates present in brain homogenate can elicit further aggregation of tau. which is first detected at 1.5 months of age (Holmes et al., Proc Natl Acad Sci U S A. 2014 Oct 14:111(41):E4376-85). Behaviorally. these mice develop motor deficits that progress to paralysis at 9 to 10 months, which is associated with deficits in nesting behavior and feeding difficulties. Greater than 50% of PS19 mice experience early mortality by 12 months of age when on a mixed background strain. In this study, non-transgenic and P301S tau transgenic littermates were given an intra-cisterna magna (ICM) dose of 6E+11vg AAV9/hTau5i-GFP per mouse or vehicle at 3 months of age. AAV9/hTau5i-GFP comprises AAV9 capsids that are packaged with self-complementary AAV genome comprising a mutant AAV2 inverted terminal repeat (ITR) with the D element deleted, the U6 promoter, the hTau5i microRNA shuttle sequence, a T6 termination signal, the CBh promoter, a GFP DNA coding sequence, the polyadenylation signal, and wild-type AAV2 ITR.

### Methods

Non-transgenic (wild-type: WT) and P301S tau transgenic male and female littermates were either ICM injected with 10 [symbol] L of vehicle (WT: n=7, 4 females, 3 males: P301S: n=6, 3 females, 3 males) or 6E+11vg AAV9/hTau5i-GFP (WT: n=8, 4 females, 4 males: P301S: n=7, 3 females, 4 males) at 3 months of age. Animals were then observed and weighed 3 times per week for 4 weeks post-dosing and then 1 time per week. Mice were monitored for clinical signs, adverse events and mortality following the treatment every week. At 3 months post-dosing, animals were necropsied for histological and biochemical analysis of the brain. Biochemical analysis included qPCR analysis of human tau (MAPT) mRNA, enzyme-linked immunoassay (ELISA) analysis of total tau protein using the Tau5 antibody and seeding assays to evaluate the presence of pathological tau species.

### Results

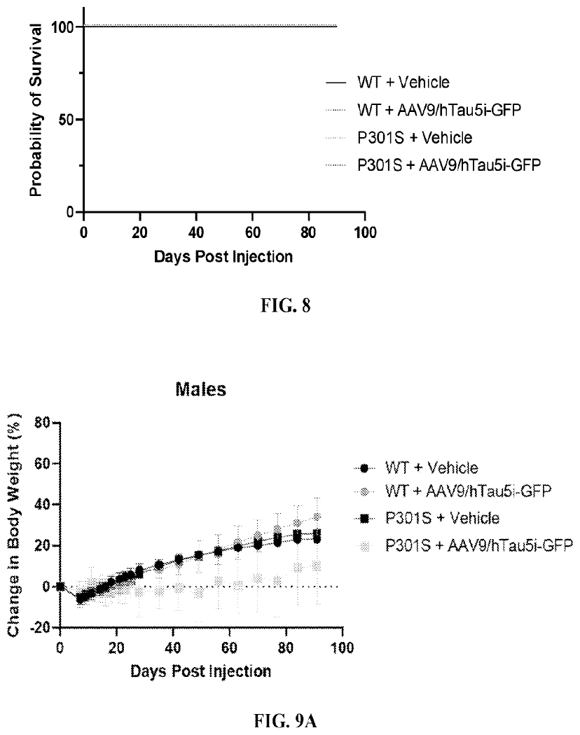

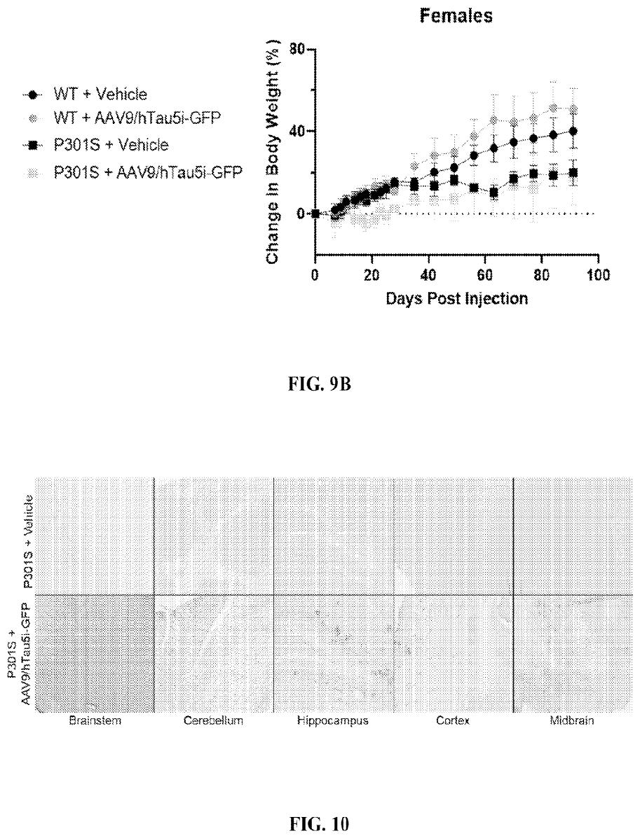

Safety of ICM delivered 6E+11vg AAV9/hTau5i-GFP is supported by survival data showing that there were no early deaths in any of the treatment groups before the planned 3 months post-dosing timepoint (FIG. 8). There were no outward signs of toxicity noted over the duration of the study. The change in body weight following injection was monitored to assess the overall health of the animals. FIGS. 9A and 9B shows that following injection there was not a significant difference in the body weight change in AAV9/hTau5i-GFP treated animals as compared to vehicle treated animals of the same genotype. Additionally, there was not a significant difference in weight gain between vehicle treated WT and P301S mice at this age (FIGS. 9A and 9B). Together, survival and body weight data support that ICM delivered dose of 6E+11vg AAV9/hTau5i-GFP was well tolerated in WT and P301S Tau littermates up to 3 months following treatment.

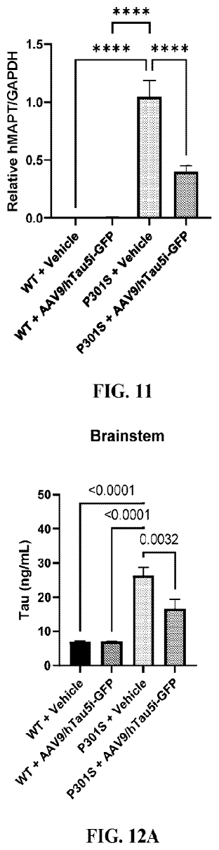

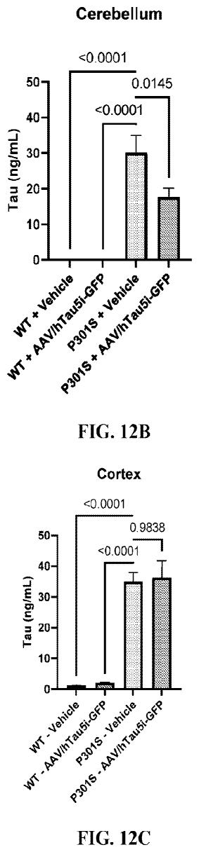

At 3 months post-injection, tissues were histologically and biochemically assessed. In FIG. 10, representative images of immunohistochemical staining using a GFP antibody show that ICM injection of AAV9/hTau5i-GFP resulted in vector distribution throughout the brain while positivity was absent in vehicle treated mice. As shown in FIG. 11, target engagement was confirmed with quantitative PCR analysis of brainstem tissue from treated mice, where an ICM delivered dose of 6E+11vg AAV9/hTau5i-GFP significantly decreased human tau mRNA levels in P301S tau mice as compared to vehicle treated P301S tau mice (One way ANOVA with Dunnett's multiple comparison test, ****p<0.0001). As expected, human tau mRNA was not significantly detected in WT littermates (FIG. 11, One way ANOVA with Dunnett's multiple comparison test, ****p<0.0001). FIG. 12 shows ELISA analysis of total tau levels in the brainstem was performed using the Tau5 antibody that detects both mouse and human tau. WT littermates had significantly less total tau as compared to P301S tau mice, which was unchanged by treatment with AAV9/hTau5i-GFP. confirming that hTau5i does not decrease mouse tau levels (FIG. 12: One-way ANOVA with Dunnett's multiple comparison test). In contrast, treatment with AAV9/hTau5i-GFP significantly decreased total tau levels in P301S tau mice in brain regions closest to the site of delivery (FIG. 12: One-way ANOVA with Dunnett's multiple comparison test. compared to P301S+Vehicle). confirming that tau protein is decreased as a result of decreased human tau mRNA.

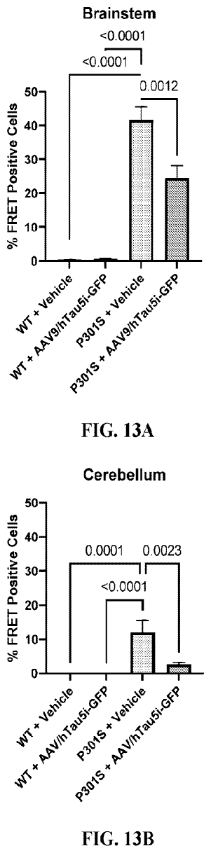

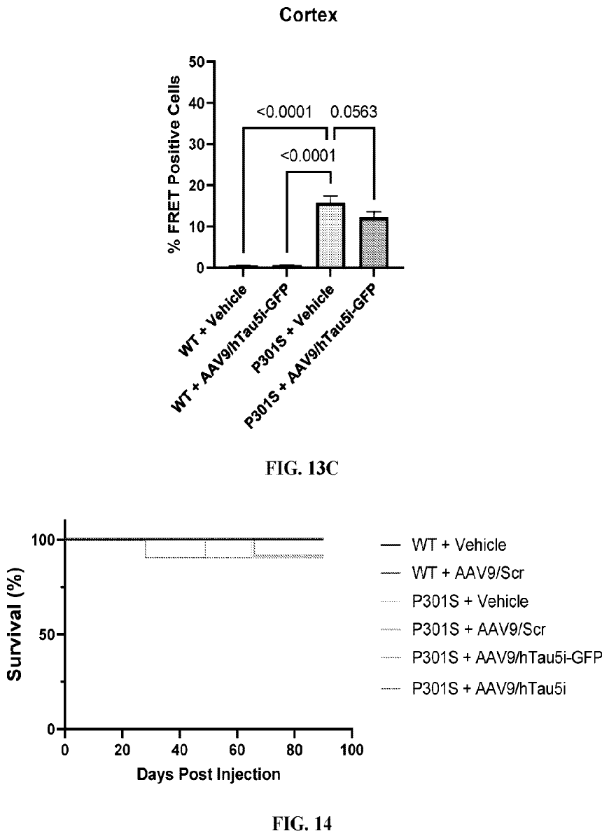

To assess the potential benefit of decreasing tau protein levels we used the tau “seeding” assay. which is an in vitro method to measure the ability of pathological tau aggregates to induce misfolding of naïve monomeric tau. In the seeding assay. HEK293 cells stably expressing the repeat domain of tau fused with a cyan fluorescent protein or yellow fluorescent protein that will create Forster resonance energy transfer (FRET) when these cells are treated with pathological tau that induces aggregate formation. FIG. 13 shows seeding assay results where transfection of brain homogenate from WT mice treated with vehicle or AAV9/hTau5i-GFP resulted in no FRET-positive inclusions and the addition of brain lysate from P301S treatment cohorts induced intracellular FRET positive aggregates (One-way ANOVA. Dunnett's multiple comparison test compared to P301S+Vehicle). Importantly. treatment with 6E+11vg AAV9/hTau5i-GFP at 3 months of age prevented tau aggregate formation when assessed at 6 months of age in the brainstem, cerebellum, and cortex (FIG. 13. One-way ANOVA. Dunnett's multiple comparison test compared to P301S+Vehicle). The decrease in pathological tau in the cortex even though that brain region was not significantly targeted by viral vector from this delivery route (FIGS. 10 and 12C). suggests that there may be benefits to distal brain regions when tau levels are decreased in connected neural networks. Together this data supports that AAV9 delivery of the hTau5i microRNA shuttle early in the disease process can selectively decrease human tau mRNA. decreasing tau protein levels and inhibiting pathogenic tau formation in the brain.

### Example 3: Study to Evaluate the Efficacy of AAV9/hTau5i when Administered Intra-Cisterna Magna in 6 Month-Old P301S Tau Mice

This study was designed to assess the therapeutic benefit of AAV9/hTauSi in the PS19 mouse model of tauopathy when administered mid-disease progression. In this study. non-transgenic and P301S tau transgenic littermates received an ICM injection of vehicle or 6E+11vg per mouse of AAV9/hTau5i-GFP. AAV9/hTau5i, or AAV9/Ser at 6 months of age. AAV9/hTau5i comprises AAV9 capsids that are packaged with self-complementary AAV genome comprising a mutant AAV2 ITR with the D element deleted, the U6 promoter, the hTau5i microRNA shuttle sequence, a T6 termination signal. the CBh promoter. the polyadenylation signal. and wild-type AAV2 ITR. AAV9/Scr comprises AAV9 capsids that are packaged with self-complementary AAV genome comprising a mutant AAV2 ITR with the D element deleted, the U6 promoter, a scrambled/non-specific microRNA shuttle sequence, a T6 termination signal, the CBh promoter, the polyadenylation signal, and wild-type AAV2 ITR. An additional purpose of this design was to perform a bridging study between the AAV9/hTau5i-GFP vector designed for preclinical studies and the AAV9/hTau5i vector, the unaltered design of which can translate directly for clinical use. The AAV9/Ser vector is an additional control for viral vector delivery and the expression of microRNAs.

### Methods

WT and P301S tau transgenic male and female littermates were ICM injected with 10 [symbol] L of vehicle (WT: n=12, 6 females, 6 males: P301S: n=11, 6 females, 5 males), 6E+11vg AAV9/Scr (WT: n=12, 6 females, 6 males: P301S: n=10, 5 females, 5 males), 6E+11vg AAV9/hTau5i-GFP (P301S: n=11, 6 females, 5 males), or 6E+11vg AAV9/hTau5i (P301S: n=11, 6 females, 5 males) at 6 months of age. Animals were then observed and weighed 3 times per week for 4 weeks post-dosing and then 1 time per week. Mice were monitored for clinical signs, adverse events and mortality following the treatment every week. At 3 months post-dosing, animals were necropsied.

### Results

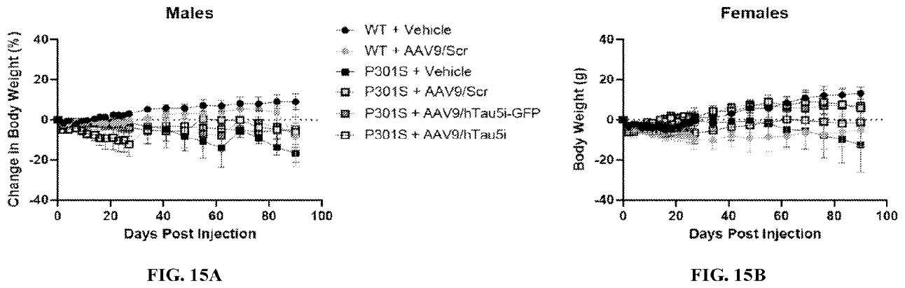

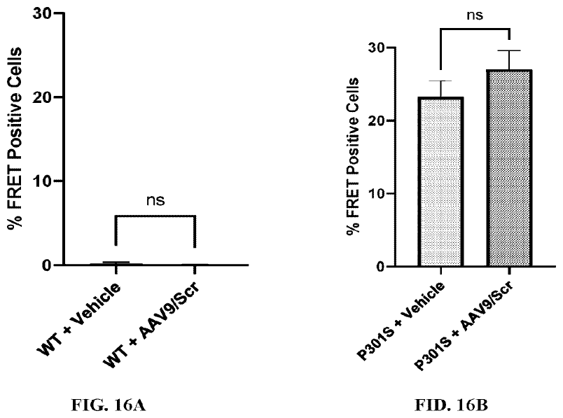

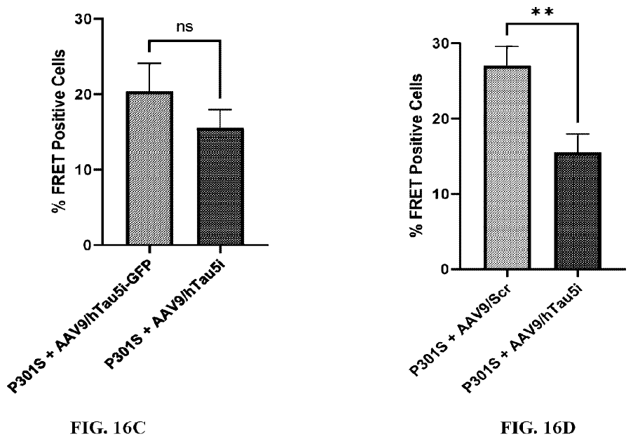

As shown in FIG. 14, there were no early deaths in the WT mice injected with vehicle or AAV9/Scr vector before the planned 3 months post-dosing timepoint. In the P301S mice, there was l early death in each of the four P301S treatment groups (FIG. 14). Together, the survival data supports that ICM delivery of either AAV9/Scr, AAV9/hTauSi-GFP or AAV9/hTauSi is not detrimental to the survival of either WT or P301S mice when treated at 6 months of age. There were no outward signs of toxicity noted over the duration of the study. The change in body weight following injection was monitored to assess the overall health of the animals. As shown in FIG. 15, at this age there was not a significant difference in changes in body weight between WT and P301S mice. Additionally, there was not a significant difference in the body weights of AAV9/Scr, AAV9/hTau5i or AAV9/hTau5i-GFP treated mice, demonstrating that all vectors were well tolerated up to 3 months following treatment in 6 month old P301S tau mice (FIG. 15.). A seeding assay was performed using brainstem lysate from treated mice. Treatment with AAV9/Scr did not change seeding activity in either WT (FIG. 16A) or P301S mice (FIG. 16B) as compared to vehicle treated animals. FIG. 16C shows that P301S mice treated with AAV9/hTau5i-GFP or AAV9/hTau5i had comparable levels of seeding. supporting that the two vectors have similar activity. Treatment with 6E+11vg AAV9/hTau5 at 6 months of age prevented tau aggregate formation in the brainstem as compared to P301S mice treated with AAV9/Scr (FIG. 16D), Student's unpaired t-test.

### Example 4: Study to Evaluate the Efficacy of AAV9/hTau5i when Administered Intra-Cisterna Magna in 9 Month-Old P301S Tau Mice

This study was designed to assess the therapeutic benefit of AAV9/hTauSi in the PS19 mouse model of tauopathy when administered late during the disease progression. In this study, non-transgenic and P301S tau transgenic littermates received an ICM dose of 6E+11vg AAV9/hTau5i or AAV9/Scr at 9 months of age.

### Methods

WT and P301S tau transgenic male and female littermates were ICM injected with 10 μL of 6E+11vg AAV9/Scr (WT: n=20, 10 females, 10 males: P301S: n=23, 15 females, 8 males) or 6E+11vg AAV9/hTau5i (P301S: n=18, 11 females, 7 males) at 9 months of age. Animals were then observed and weighed 3 times per week for 4 weeks post-dosing and then I time per week. Mice were monitored for clinical signs, adverse events and mortality following the treatment every week. At 3 months post-dosing, animals were necropsied.

### Results

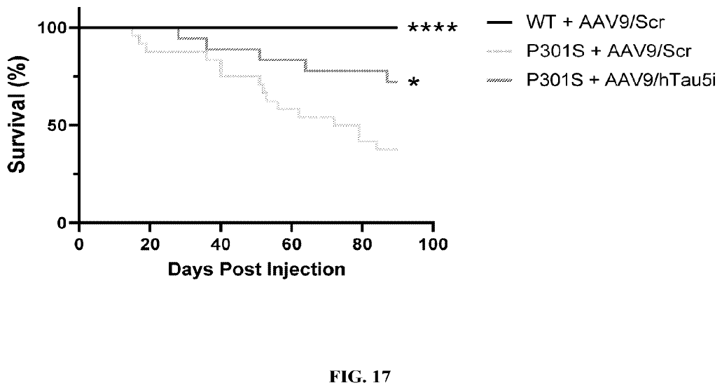

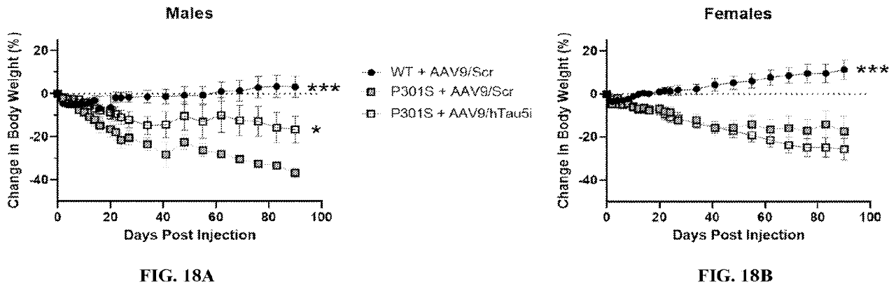

As shown in FIG. 17, compared to WT littermates injected with AAV9/Ser, where 100% of animals survived to the study endpoint, P301S mice injected with AAV9/Ser had a significantly lower survival and only 39% survived study endpoint (Mantel-Cox, ****p<0.0001). Despite the late age of treatment, ICM injection of 6E+11vg AAV9/hTau5i significantly prolonged survival of P301S mice to 72% at the study endpoint (FIG. 17, Mantel-Cox, *p<0.05). The change in body weight following injection was monitored to assess the overall health of the animals. As shown in FIG. 18, at this late stage of the disease progress, AAV9/Scr treated P301S male and female mice had significantly decreased changes in body weight as compared to AAV9/Scr WT mice (Two-way ANOVA, Sidak's multiple comparison test, ***p<0.001). Treatment with AAV9/hTau5i significantly slowed loss of body weight in P301S males and did not affect body weight loss in P301S females as compared to AAV9/Scr treated P301S sex-matched mice (FIG. 18; two-way ANOVA, Sidak's multiple comparison test. *p<0.05). Together, this in-life data supports a potential treatment benefit of AAV9/hTau5i for tauopathies, even when provided late in the disease course.

### Example 5: Study to Evaluate the Safety of AAV9/hTau5i when Administered Intra-Cisterna Magna in 3-Month-Old WT C57BL/6J Mice

This study was designed to assess the safety of reducing endogenous tau levels in mice. In this study, WT C57BL/6J mice received an ICM dose of vehicle or 4.7E+11vg AAV9/mTau2i or AAV9/Scr at 3 months of age.

### Methods

WT C57BL/6J mice male and female littermates were ICM injected with 10 μL of vehicle (n=30, 15 females, 5 males), 4.7E+11vg AAV9/Scr (n=30, 15 females, 15 males) or 4.7E+11vg AAV9/mTau2i (n=30, 15 females, 15 males) at 3 months of age. Mice were monitored for clinical signs, adverse events and mortality following the treatment every week. An interim group of animals (n=10, 5 females, 5 males per treatment group) were harvested at 1-month post-injection for clinical blood chemistry and histopathology analysis. Remaining animals underwent behavioral testing at 1-month post-dosing and are aging to study-endpoint, which is 12 months post-dosing.

### Results

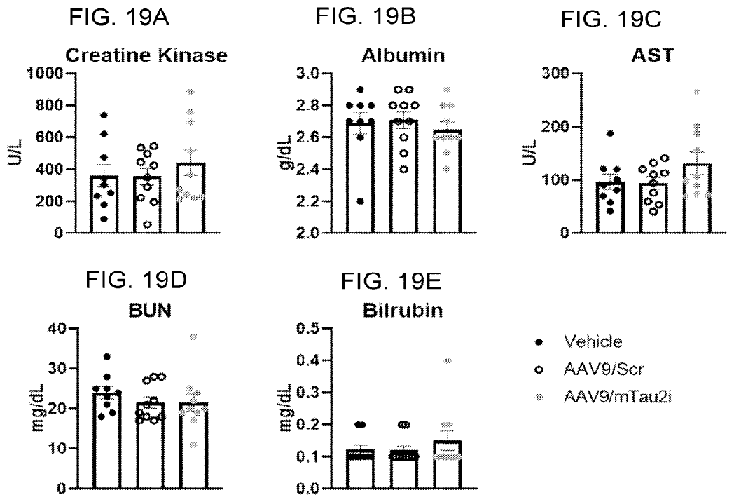

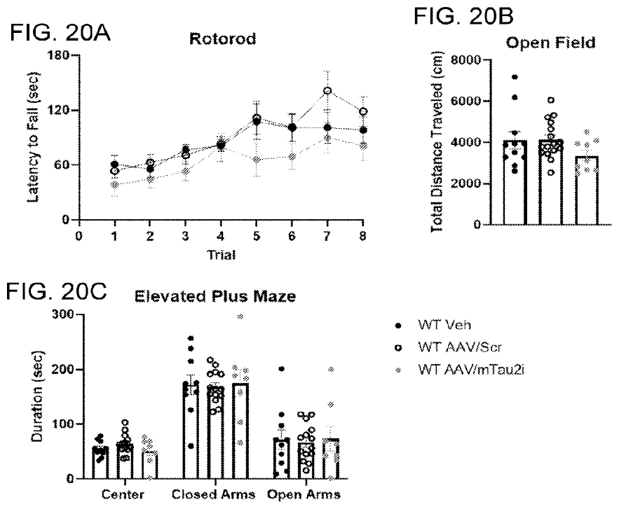

To date, no difference in clinical signs or mortality have been found between treatment groups. Tissues from the interim group were histologically normal and as shown in FIG. 19. The group aging to study endpoint underwent behavioral assessment for motor learning and coordination (Rotarod), general activity (Open Field) and anxiety (Elevated Plus Maze) at 1-month post-injection. FIG. 20 shows that there were no significant changes in behavior in these three tasks with knock-down of endogenous tau in the AAV9/m Tau2i treated group compared to vehicle and AAV9/Scr treated mice. Together these data support that knock-down of endogenous tau is well-tolerated in the short term.

What is claimed is:

1. A recombinant adeno-associated virus (rAAV) vector comprising at least one polynucleotide sequence encoding at least one artificial micro RNA (amiRNA) directed against MAPT, wherein the amiRNA directed against MAPT comprises the nucleic acid sequence set forth in any one of SEQ ID NOs: 139-186.

2. The rAAV vector of claims 1, wherein the polynucleotide sequence encoding the at least one amiRNA directed against MAPT comprises the nucleic acid sequence set forth in any one of SEQ ID NOs: 41-88.

3. An rAAV vector of claim 1 or 2, wherein the rAAV vector further comprises a first inverted terminal repeat (ITR) sequence, wherein the first ITR sequence is an AAV2 ITR sequence.

4. The rAAV vector of claim 3, wherein the first ITR sequence comprises the sequence set forth in SEQ ID NO: 15.

5. An rAAV vector of any one of claim 1-4 wherein the rAAV vector further comprises a second ITR sequence, wherein the second ITR sequence is an AAV2 ITR sequence.

6. The rAAV vector of claim 5, wherein the second ITR sequence comprises the sequence set forth in SEQ ID NO: 16.

7. The rAAV vector of any one of claims 1-6, wherein the rAAV vector further comprises a first promoter sequence, wherein the first promoter sequence is a murine U6 promoter sequence.

8. The rAAV vector of claim 7, wherein the murine U6 promoter sequence comprises the nucleic acid sequence set forth in SEQ ID NO: 31.

9. The rAAV vector of any one of claims 1-8, wherein the rAAV vector further comprises a second promoter sequence, wherein the second promoter sequence is a chicken β-actin hybrid (CBh) promoter sequence.

10. The rAAV vector of claim 9, wherein the CBh promoter sequence comprises the nucleic acid sequence set forth in SEQ ID NO: 29.

11. The rAAV vector of any one of claims 1-10, wherein the rAAV vector further comprises a termination signal.

12. The rAAV vector of claim 11, wherein the termination signal comprises the nucleic acid sequence set forth in SEQ ID NO: 40.

13. An rAAV vector comprising, in the 5′ to 3′ direction:
a. a first AAV2 ITR sequence;
b. a murine U6 promoter sequence;
c. a polynucleotide sequence encoding for at least one amiRNA directed against MAPT, wherein the amiRNA directed against MAPT comprises the nucleic acid sequence set forth in any one of SEQ ID NOs: 139-186;
d. a termination sequence;
e. a CBh promoter sequence;
f. a synthetic polyA sequence; and
g. a second AAV2 ITR sequence.

14. The rAAV vector of claim 13, wherein the rAAV vector comprises the sequence set forth in SEQ ID NO: 14.

15. An rAAV viral vector comprising:
(a) an AAV capsid protein; and
(b) an rAAV vector of any one of claims 1-14.

16. The rAAV viral vector of claim 15, wherein the AAV capsid protein is an AAV9 capsid protein.

17. A pharmaceutical composition comprising the rAAV vector of any one of claims 1-14 or the rAAV viral vector of claim 15 or 16; and at least one pharmaceutically acceptable excipient and/or additive.

18. A method for treating a tauopathy in a subject in need thereof, the method comprising administering to the subject a therapeutically effective amount of the rAAV vector of any one of claims 1-14, the rAAV viral vector of claim 15 or 16, or the pharmaceutical composition of claim 17.

19. The method of claim 18, wherein the tauopathy is Alzheimer's Disease or Frontotemporal Dementia and Parkinsonism Linked To Chromosome 17 (FTDP-17).

20. The method of claim 18 or 19, wherein the subject has one or more mutations in the MAPT gene.

21. The rAAV vector of any one of claims 1-14, the rAAV viral vector of claim 15 or 16, or the pharmaceutical composition of claim 17 for use in the treatment of a tauopathy.

22. The rAAV vector, the rAAV viral vector, or the pharmaceutical compositions for use of claims 21, wherein the tauopathy is Alzheimer's Disease or FTDP-17.

23. The rAAV vector, the rAAV viral vector, or the pharmaceutical compositions of claim 21 or 22, wherein the tauopathy is associated with one or more mutations in the MAPT gene.


SEQUENCE LISTING (193 sequences)

SEQ ID NO: 1 - DNA, 80 bp, Artificial Sequence
  sequence encoding MAPT-directed miRNA
gagtgagcgccgccaccaggattccagcaaactgtaaagccacagatgggtttgctggaatcctggtggcgttgcctact

SEQ ID NO: 2 - DNA, 6590 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 3 - DNA, 5639 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 4 - DNA, 5465 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 5 - DNA, 5372 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 6 - DNA, 5552 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 7 - DNA, 6644 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 8 - DNA, 5459 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 9 - DNA, 5546 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 10 - DNA, 6815 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 11 - DNA, 6524 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 12 - DNA, 4139 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 13 - DNA, 5525 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 14 - DNA, 5168 bp, Artificial Sequence
  rAAV construct comprising hTau5i
ctgcgcgctcgctcgctcactgaggccgcccgggcaaagcccgggcgtcgggcgacctttggtcgcccggcctcagtgagcgagcgagcgcgcagagagggagtggggttcggtacccctagggacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttgcgtttagtgaaccgtcagatggtaccgtttaaactcgagtgagcgccgccaccaggattccagcaaactgtaaagccacagatgggtttgctggaatcctggtggcgttgcctactagagcggccgccacagcggggagatccagacatgataagatacattttttgaattcagatccgagctcggtaccaagcttggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggacctaataaagagctcagatgcatcgatcagagtgtgttggttttttgtgtgacgcgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccctgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 15 - DNA, 106 bp, Artificial
  ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 16 - DNA, 169 bp, Artificial
  ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttc

SEQ ID NO: 17 - DNA, 106 bp, Artificial
  ITR
ctgcgcgctcgctcgctcactgaggccgccggcccaaagggccggcgtcgggcgacctttggtcgcccggcctcagtgagcgagcgagcgcgcagagagggagtgg

SEQ ID NO: 18 - DNA, 145 bp, Artificial
  ITR
aggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaa

SEQ ID NO: 19 - DNA, 170 bp, Artificial
  ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttc

SEQ ID NO: 20 - DNA, 104 bp, Artificial
  ITR
tgcgcgctcgctcgctcactgaggccgcccgggcaaagcccgggcgtcgggcgacctttggtcgcccggcctcagtgagcgagcgagcgcgcagagagggagtg

SEQ ID NO: 21 - DNA, 106 bp, Artificial; identical to SEQ ID NO: 17
  ITR

SEQ ID NO: 22 - DNA, 130 bp, Artificial
  ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 145 bp, Artificial
  ITR
aggaacccctagtgatggagttggccactccctctctgcgcgctcgctcgctcactgaggccgccggcccaaagggccggcgtcgggcgacctttggtcgcccggcctcagtgagcgagcgagcgcgcagagagggagtggccaa

SEQ ID NO: 24 - DNA, 145 bp, Artificial; identical to SEQ ID NO: 18
  ITR

SEQ ID NO: 25 - DNA, 164 bp, Artificial
  JeT Promoter
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgt

SEQ ID NO: 26 - DNA, 225 bp, Artificial
  MeP229 promoter
caattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 27 - DNA, 426 bp, Artificial
  MeP426 promoter
ataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaa

SEQ ID NO: 28 - DNA, 793 bp, Artificial
  CBh promoter
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 29 - DNA, 797 bp, Artificial
  CBh promoter
tacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggac

SEQ ID NO: 30 - DNA, 241 bp, Artificial
  U6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 31 - DNA, 311 bp, Artificial
  mU6 promoter
gacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 32 - DNA, 105 bp, Artificial
  prokaryotic promoter
cgcggaacccctatttgtttatttttctaaatacattcaaatatgtatccgctcatgagacaataaccctgataaatgcttcaataatattgaaaaaggaagagt

SEQ ID NO: 33 - DNA, 123 bp, Artificial
  SV40 polyA
tgtttattgcagcttataatggttacaaataaagcaatagcatcacaaatttcacaaataaagcatttttttcactgcattctagttgtggtttgtccaaactcatcaatgtatcttatcatg

SEQ ID NO: 34 - DNA, 254 bp, Artificial
  BGH polyA
ctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatggcttctgaggcggaaagaaccagct

SEQ ID NO: 35 - DNA, 225 bp, Artificial
  BGH polyA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatgg

SEQ ID NO: 36 - DNA, 48 bp, Artificial
  Synthetic PolyA
aataaagagctcagatgcatcgatcagagtgtgttggttttttgtgtg

SEQ ID NO: 37 - DNA, 105 bp, Artificial; identical to SEQ ID NO: 32
  AmpR promoter

SEQ ID NO: 38 - DNA, 816 bp, Artificial
  Kanamycin Resistance
atgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgtgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaa

SEQ ID NO: 39 - DNA, 589 bp, Artificial
  Origin of Replication
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 40 - DNA, 6 bp, Artificial
  T6 termination sequence
tttttt

SEQ ID NO: 41 - DNA, 80 bp, Artificial
  hTau1i miRNA Shuttle DNA
gagtgagcgaccgccaggagttcgaagtgatctgtaaagccacagatgggatcacttcgaactcctggcgggtgcctact

SEQ ID NO: 42 - DNA, 80 bp, Artificial
  hTau2i miRNA Shuttle DNA
gagtgagcgccgctggcctgaaagctgaagactgtaaagccacagatgggtcttcagctttcaggccagcgttgcctact

SEQ ID NO: 43 - DNA, 80 bp, Artificial
  hTau3i miRNA Shuttle DNA
gagtgagcgccgggactggaagcgatgacaactgtaaagccacagatgggttgtcatcgcttccagtcccgttgcctact

SEQ ID NO: 44 - DNA, 80 bp, Artificial
  hTau4i miRNA Shuttle DNA
gagtgagcgatggcggaggaaataaaaagatctgtaaagccacagatgggatctttttatttcctccgccagtgcctact

SEQ ID NO: 45 - DNA, 80 bp, Artificial; identical to SEQ ID NO: 1
  hTau5i miRNA Shuttle DNA

SEQ ID NO: 46 - DNA, 80 bp, Artificial
  hTau6i miRNA Shuttle DNA
gagtgagcgctcgcatggtcagtaaaagcaactgtaaagccacagatgggttgcttttactgaccatgcgagtgcctact

SEQ ID NO: 47 - DNA, 80 bp, Artificial
  hTau7i miRNA Shuttle DNA
gagtgagcgagagttcgaagtgatggaagatctgtaaagccacagatgggatcttccatcacttcgaactcctgcctact

SEQ ID NO: 48 - DNA, 80 bp, Artificial
  hTau8i miRNA Shuttle DNA
gagtgagcgccccaagctcgcatggtcagtactgtaaagccacagatgggtactgaccatgcgagcttgggttgcctact

SEQ ID NO: 49 - DNA, 80 bp, Artificial
  hTau9i miRNA Shuttle DNA
gagtgagcgcgccaagggggctgatggtaaactgtaaagccacagatgggtttaccatcagcccccttggcttgcctact

SEQ ID NO: 50 - DNA, 80 bp, Artificial
  hTau10i miRNA Shuttle DNA
gagtgagcgacggcagcatcgacatggtagactgtaaagccacagatgggtctaccatgtcgatgctgccggtgcctact

SEQ ID NO: 51 - DNA, 80 bp, Artificial
  hTau11i miRNA Shuttle DNA
gagtgagcgccggggcggagatcgtgtacaactgtaaagccacagatgggttgtacacgatctccgccccgttgcctact

SEQ ID NO: 52 - DNA, 80 bp, Artificial
  mTau1i miRNA Shuttle DNA
gagtgagcgactcgccaggagtttgacacaactgtaaagccacagatgggttgtgtcaaactcctggcgaggtgcctact

SEQ ID NO: 53 - DNA, 80 bp, Artificial
  mTau2i miRNA Shuttle DNA
gagtgagcgatcgccaggagtttgacacaatctgtaaagccacagatgggattgtgtcaaactcctggcgagtgcctact

SEQ ID NO: 54 - DNA, 80 bp, Artificial
  mTau3i miRNA Shuttle DNA
gagtgagcgccgccaggagtttgacacaatgctgtaaagccacagatgggcattgtgtcaaactcctggcgatgcctact

SEQ ID NO: 55 - DNA, 80 bp, Artificial
  mTau4i miRNA Shuttle DNA
gagtgagcgagagacatggaccatggcttaactgtaaagccacagatgggttaagccatggtccatgtctcctgcctact

SEQ ID NO: 56 - DNA, 80 bp, Artificial
  mTau5i miRNA Shuttle DNA
gagtgagcgcgccgctgggcatgtgactcaactgtaaagccacagatgggttgagtcacatgcccagcggcttgcctact

SEQ ID NO: 57 - DNA, 80 bp, Artificial
  mTau6i miRNA Shuttle DNA
gagtgagcgacagcaaagacaggacaggaaactgtaaagccacagatgggtttcctgtcctgtctttgctggtgcctact

SEQ ID NO: 58 - DNA, 80 bp, Artificial
  mTau7i miRNA Shuttle DNA
gagtgagcgagagcagaaattgtgtataagtctgtaaagccacagatgggacttatacacaatttctgctcctgcctact

SEQ ID NO: 59 - DNA, 80 bp, Artificial
  mTau8i miRNA Shuttle DNA
gagtgagcgaaccttcagggagaatgccaaactgtaaagccacagatgggtttggcattctccctgaaggtctgcctact

SEQ ID NO: 60 - DNA, 80 bp, Artificial
  mTau9i miRNA Shuttle DNA
gagtgagcgaagcaaagacaggacaggaaatctgtaaagccacagatgggatttcctgtcctgtctttgctgtgcctact

SEQ ID NO: 61 - DNA, 80 bp, Artificial
  mTau10i miRNA Shuttle DNA
gagtgagcgaagggtcaggtgaaccaccaaactgtaaagccacagatgggtttggtggttcacctgaccctgtgcctact

SEQ ID NO: 62 - DNA, 80 bp, Artificial
  mTau11i miRNA Shuttle DNA
gagtgagcgagacaggaaatgacgagaagaactgtaaagccacagatgggttcttctcgtcatttcctgtcctgcctact

SEQ ID NO: 63 - DNA, 80 bp, Artificial
  mTau12i miRNA Shuttle DNA
gagtgagcgcgggtcaggtgaaccaccaaaactgtaaagccacagatgggttttggtggttcacctgacccttgcctact

SEQ ID NO: 64 - DNA, 80 bp, Artificial
  mTau13i miRNA Shuttle DNA
gagtgagcgaggtcaggtgaaccaccaaaatctgtaaagccacagatgggattttggtggttcacctgaccctgcctact

SEQ ID NO: 65 - DNA, 80 bp, Artificial
  mTau14i miRNA Shuttle DNA
gagtgagcgagtcaggtgaaccaccaaaatcctgtaaagccacagatggggattttggtggttcacctgacctgcctact

SEQ ID NO: 66 - DNA, 80 bp, Artificial
  mTau15i miRNA Shuttle DNA
gagtgagcgcccggagaacgaagcggctacactgtaaagccacagatgggtgtagccgcttcgttctccggatgcctact

SEQ ID NO: 67 - DNA, 80 bp, Artificial
  mTau16i miRNA Shuttle DNA
gagtgagcgacctaagtcaccatcagctagtctgtaaagccacagatgggactagctgatggtgacttagggtgcctact

SEQ ID NO: 68 - DNA, 80 bp, Artificial
  mTau17i miRNA Shuttle DNA
gagtgagcgaaggtcgaagattggctctactctgtaaagccacagatgggagtagagccaatcttcgacctgtgcctact

SEQ ID NO: 69 - DNA, 80 bp, Artificial
  mTau18i miRNA Shuttle DNA
gagtgagcgacaggaggtggcaaggtgcaaactgtaaagccacagatgggtttgcaccttgccacctcctggtgcctact

SEQ ID NO: 70 - DNA, 80 bp, Artificial
  mTau19i miRNA Shuttle DNA
gagtgagcgaaggaggtggcaaggtgcaaatctgtaaagccacagatgggatttgcaccttgccacctcctgtgcctact

SEQ ID NO: 71 - DNA, 80 bp, Artificial
  hmTau1i miRNA Shuttle DNA
gagtgagcgatccacggcacctcagcaatgtctgtaaagccacagatgggacattgctgaggtgccgtggagtgcctact

SEQ ID NO: 72 - DNA, 80 bp, Artificial
  hmTau2i miRNA Shuttle DNA
gagtgagcgagagggaataagaagattgaaactgtaaagccacagatgggtttcaatcttcttattccctcctgcctact

SEQ ID NO: 73 - DNA, 80 bp, Artificial
  hmTau3i miRNA Shuttle DNA
gagtgagcgagacagagtccagtcgaagattctgtaaagccacagatgggaatcttcgactggactctgtcctgcctact

SEQ ID NO: 74 - DNA, 80 bp, Artificial
  hmTau4i miRNA Shuttle DNA
gagtgagcgatggccaggtggaagtaaaatcctgtaaagccacagatggggattttacttccacctggccactgcctact

SEQ ID NO: 75 - DNA, 80 bp, Artificial
  hmTau5i miRNA Shuttle DNA
gagtgagcgagtggccaggtggaagtaaaatctgtaaagccacagatgggattttacttccacctggccacctgcctact

SEQ ID NO: 76 - DNA, 80 bp, Artificial
  hmTau6i miRNA Shuttle DNA
gagtgagcgcggccaggtggaagtaaaatcactgtaaagccacagatgggtgattttacttccacctggccatgcctact

SEQ ID NO: 77 - DNA, 80 bp, artificial
  hmTau7i miRNA Shuttle DNA
gagtgagcgagaggtggccaggtggaagtaactgtaaagccacagatgggttacttccacctggccacctcctgcctact

SEQ ID NO: 78 - DNA, 80 bp, Artificial
  hmTau8i miRNA Shuttle DNA
gagtgagcgactcgttagggaacatccatcactgtaaagccacagatgggtgatggatgttccctaacgagctgcctact

SEQ ID NO: 79 - DNA, 80 bp, Artificial
  hmTau9i miRNA Shuttle DNA
gagtgagcgatccacggcatctcagcaatgtctgtaaagccacagatgggacattgctgagatgccgtggagtgcctact

SEQ ID NO: 80 - DNA, 80 bp, Artificial
  hmTau10i miRNA Shuttle DNA
gagtgagcgactcattaggcaacatccatcactgtaaagccacagatgggtgatggatgttgcctaatgagctgcctact

SEQ ID NO: 81 - DNA, 80 bp, Artificial
  hmTau11i miRNA Shuttle DNA
gagtgagcgactgtgcccatgccagacctaactgtaaagccacagatgggttaggtctggcatgggcacaggtgcctact

SEQ ID NO: 82 - DNA, 80 bp, Artificial
  hmTau12i miRNA Shuttle DNA
gagtgagcgatgcccatgccagacctaaagactgtaaagccacagatgggtctttaggtctggcatgggcactgcctact

SEQ ID NO: 83 - DNA, 80 bp, Artificial
  hmTau13i miRNA Shuttle DNA
gagtgagcgcgcccatgccagacctaaagaactgtaaagccacagatgggttctttaggtctggcatgggcatgcctact

SEQ ID NO: 84 - DNA, 80 bp, Artificial
  hmTau14i miRNA Shuttle DNA
gagtgagcgacccatgccagacctaaagaatctgtaaagccacagatgggattctttaggtctggcatgggctgcctact

SEQ ID NO: 85 - DNA, 80 bp, Artificial
  hmTau15i miRNA Shuttle DNA
gagtgagcgaccatgccagacctaaagaatgctgtaaagccacagatgggcattctttaggtctggcatgggtgcctact

SEQ ID NO: 86 - DNA, 80 bp, artificial
  hmTau16i miRNA Shuttle DNA
gagtgagcgagcaaggtgcaaatagtctacactgtaaagccacagatgggtgtagactatttgcaccttgcctgcctact

SEQ ID NO: 87 - DNA, 80 bp, Artificial
  hmTau17i miRNA Shuttle DNA
gagtgagcgaacctccaagtgtggctcgttactgtaaagccacagatgggtaacgagccacacttggaggtctgcctact

SEQ ID NO: 88 - DNA, 80 bp, Artificial
  hmTau18i miRNA Shuttle DNA
gagtgagcgagcgagaacgccaaagccaagactgtaaagccacagatgggtcttggctttggcgttctcgcgtgcctact

SEQ ID NO: 89 - DNA, 80 bp, Artificial
  Scramble miRNA Shuttle DNA
gagtgagcgaccgctaagcatgcaccgttctctgtaaagccacagatgggatcacttcgaactcctggcgggtgcctact

SEQ ID NO: 90 - RNA, 80 nt, Artificial
  hTau1i Full length miRNA Sequence (5′-3′)
gagugagcgaccgccaggaguucgaagugaucuguaaagccacagaugggaucacuucgaacuccuggcgggugccuacu

SEQ ID NO: 91 - RNA, 80 nt, Artificial
  hTau2i Full length miRNA Sequence (5′-3′)
gagugagcgccgcuggccugaaagcugaagacuguaaagccacagaugggucuucagcuuucaggccagcguugccuacu

SEQ ID NO: 92 - RNA, 80 nt, Artificial
  hTau3i Full length miRNA Sequence (5′-3′)
gagugagcgccgggacuggaagcgaugacaacuguaaagccacagauggguugucaucgcuuccagucccguugccuacu

SEQ ID NO: 93 - RNA, 80 nt, Artificial
  hTau4i Full length miRNA Sequence (5′-3′)
gagugagcgauggcggaggaaauaaaaagaucuguaaagccacagaugggaucuuuuuauuuccuccgccagugccuacu

SEQ ID NO: 94 - RNA, 80 nt, Artificial
  hTau5i Full length miRNA Sequence (5′-3′)
gagugagcgccgccaccaggauuccagcaaacuguaaagccacagauggguuugcuggaauccugguggcguugccuacu

SEQ ID NO: 95 - RNA, 80 nt, Artificial
  hTau6i Full length miRNA Sequence (5′-3′)
gagugagcgcucgcauggucaguaaaagcaacuguaaagccacagauggguugcuuuuacugaccaugcgagugccuacu

SEQ ID NO: 96 - RNA, 80 nt, Artificial
  hTau7i Full length miRNA Sequence (5′-3′)
gagugagcgagaguucgaagugauggaagaucuguaaagccacagaugggaucuuccaucacuucgaacuccugccuacu

SEQ ID NO: 97 - RNA, 80 nt, Artificial
  hTau8i Full length miRNA Sequence (5′-3′)
gagugagcgccccaagcucgcauggucaguacuguaaagccacagauggguacugaccaugcgagcuuggguugccuacu

SEQ ID NO: 98 - RNA, 80 nt, Artificial
  hTau9i Full length miRNA Sequence (5′-3′)
gagugagcgcgccaagggggcugaugguaaacuguaaagccacagauggguuuaccaucagcccccuuggcuugccuacu

SEQ ID NO: 99 - RNA, 80 nt, Artificial
  hTau10i Full length miRNA Sequence (5′-3′)
gagugagcgacggcagcaucgacaugguagacuguaaagccacagaugggucuaccaugucgaugcugccggugccuacu

SEQ ID NO: 100 - RNA, 80 nt, Artificial
  hTau11i Full length miRNA Sequence (5′-3′)
gagugagcgccggggcggagaucguguacaacuguaaagccacagauggguuguacacgaucuccgccccguugccuacu

SEQ ID NO: 101 - RNA, 80 nt, Artificial
  mTau1i Full length miRNA Sequence (5′-3′)
gagugagcgacucgccaggaguuugacacaacuguaaagccacagauggguugugucaaacuccuggcgaggugccuacu

SEQ ID NO: 102 - RNA, 80 nt, Artificial
  mTau2i Full length miRNA Sequence (5′-3′)
gagugagcgaucgccaggaguuugacacaaucuguaaagccacagaugggauugugucaaacuccuggcgagugccuacu

SEQ ID NO: 103 - RNA, 80 nt, Artificial
  mTau3i Full length miRNA Sequence (5′-3′)
gagugagcgccgccaggaguuugacacaaugcuguaaagccacagaugggcauugugucaaacuccuggcgaugccuacu

SEQ ID NO: 104 - RNA, 80 nt, Artificial
  mTau4i Full length miRNA Sequence (5′-3′)
gagugagcgagagacauggaccauggcuuaacuguaaagccacagauggguuaagccaugguccaugucuccugccuacu

SEQ ID NO: 105 - RNA, 80 nt, Artificial
  mTau5i Full length miRNA Sequence (5′-3′)
gagugagcgcgccgcugggcaugugacucaacuguaaagccacagauggguugagucacaugcccagcggcuugccuacu

SEQ ID NO: 106 - RNA, 80 nt, Artificial
  mTau6i Full length miRNA Sequence (5′-3′)
gagugagcgacagcaaagacaggacaggaaacuguaaagccacagauggguuuccuguccugucuuugcuggugccuacu

SEQ ID NO: 107 - RNA, 80 nt, Artificial
  mTau7i Full length miRNA Sequence (5′-3′)
gagugagcgagagcagaaauuguguauaagucuguaaagccacagaugggacuuauacacaauuucugcuccugccuacu

SEQ ID NO: 108 - RNA, 80 nt, Artificial
  mTau8i Full length miRNA Sequence (5′-3′)
gagugagcgaaccuucagggagaaugccaaacuguaaagccacagauggguuuggcauucucccugaaggucugccuacu

SEQ ID NO: 109 - RNA, 80 nt, Artificial
  mTau9i Full length miRNA Sequence (5′-3′)
gagugagcgaagcaaagacaggacaggaaaucuguaaagccacagaugggauuuccuguccugucuuugcugugccuacu

SEQ ID NO: 110 - RNA, 80 nt, Artificial
  mTau10i Full length miRNA Sequence (5′-3′)
gagugagcgaagggucaggugaaccaccaaacuguaaagccacagauggguuuggugguucaccugacccugugccuacu

SEQ ID NO: 111 - RNA, 80 nt, Artificial
  mTau11i Full length miRNA Sequence (5′-3′)
gagugagcgagacaggaaaugacgagaagaacuguaaagccacagauggguucuucucgucauuuccuguccugccuacu

SEQ ID NO: 112 - RNA, 80 nt, Artificial
  mTau12i Full length miRNA Sequence (5′-3′)
gagugagcgcgggucaggugaaccaccaaaacuguaaagccacagauggguuuuggugguucaccugacccuugccuacu

SEQ ID NO: 113 - RNA, 80 nt, Artificial
  mTau13i Full length miRNA Sequence (5′-3′)
gagugagcgaggucaggugaaccaccaaaaucuguaaagccacagaugggauuuuggugguucaccugacccugccuacu

SEQ ID NO: 114 - RNA, 80 nt, Artificial
  mTau14i Full length miRNA Sequence (5′-3′)
gagugagcgagucaggugaaccaccaaaauccuguaaagccacagauggggauuuuggugguucaccugaccugccuacu

SEQ ID NO: 115 - RNA, 80 nt, Artificial
  mTau15i Full length miRNA Sequence (5′-3′)
gagugagcgcccggagaacgaagcggcuacacuguaaagccacagauggguguagccgcuucguucuccggaugccuacu

SEQ ID NO: 116 - RNA, 80 nt, Artificial
  mTau16i Full length miRNA Sequence (5′-3′)
gagugagcgaccuaagucaccaucagcuagucuguaaagccacagaugggacuagcugauggugacuuagggugccuacu

SEQ ID NO: 117 - RNA, 80 nt, Artificial
  mTau17i Full length miRNA Sequence (5′-3′)
gagugagcgaaggucgaagauuggcucuacucuguaaagccacagaugggaguagagccaaucuucgaccugugccuacu

SEQ ID NO: 118 - RNA, 80 nt, Artificial
  mTau18i Full length miRNA Sequence (5′-3′)
gagugagcgacaggagguggcaaggugcaaacuguaaagccacagauggguuugcaccuugccaccuccuggugccuacu

SEQ ID NO: 119 - RNA, 80 nt, Artificial
  mTau19i Full length miRNA Sequence (5′-3′)
gagugagcgaaggagguggcaaggugcaaaucuguaaagccacagaugggauuugcaccuugccaccuccugugccuacu

SEQ ID NO: 120 - RNA, 80 nt, Artificial
  hmTau1i Full length miRNA Sequence (5′-3′)
gagugagcgauccacggcaccucagcaaugucuguaaagccacagaugggacauugcugaggugccguggagugccuacu

SEQ ID NO: 121 - RNA, 80 nt, Artificial
  hmTau2i Full length miRNA Sequence (5′-3′)
gagugagcgagagggaauaagaagauugaaacuguaaagccacagauggguuucaaucuucuuauucccuccugccuacu

SEQ ID NO: 122 - RNA, 80 nt, Artificial
  hmTau3i Full length miRNA Sequence (5′-3′)
gagugagcgagacagaguccagucgaagauucuguaaagccacagaugggaaucuucgacuggacucuguccugccuacu

SEQ ID NO: 123 - RNA, 80 nt, Artificial
  hmTau4i Full length miRNA Sequence (5′-3′)
gagugagcgauggccagguggaaguaaaauccuguaaagccacagauggggauuuuacuuccaccuggccacugccuacu

SEQ ID NO: 124 - RNA, 80 nt, Artificial
  hmTau5i Full length miRNA Sequence (5′-3′)
gagugagcgaguggccagguggaaguaaaaucuguaaagccacagaugggauuuuacuuccaccuggccaccugccuacu

SEQ ID NO: 125 - RNA, 80 nt, Artificial
  hmTau6i Full length miRNA Sequence (5′-3′)
gagugagcgcggccagguggaaguaaaaucacuguaaagccacagaugggugauuuuacuuccaccuggccaugccuacu

SEQ ID NO: 126 - RNA, 80 nt, Artificial
  hmTau7i Full length miRNA Sequence (5′-3′)
gagugagcgagagguggccagguggaaguaacuguaaagccacagauggguuacuuccaccuggccaccuccugccuacu

SEQ ID NO: 127 - RNA, 80 nt, Artificial
  hmTau8i Full length miRNA Sequence (5′-3′)
gagugagcgacucguuagggaacauccaucacuguaaagccacagaugggugauggauguucccuaacgagcugccuacu

SEQ ID NO: 128 - RNA, 80 nt, Artificial
  hmTau9i Full length miRNA Sequence (5′-3′)
gagugagcgauccacggcaucucagcaaugucuguaaagccacagaugggacauugcugagaugccguggagugccuacu

SEQ ID NO: 129 - RNA, 80 nt, Artificial
  hmTau10i Full length miRNA Sequence (5′-3′)
gagugagcgacucauuaggcaacauccaucacuguaaagccacagaugggugauggauguugccuaaugagcugccuacu

SEQ ID NO: 130 - RNA, 80 nt, artificial
  hmTau11i Full length miRNA Sequence (5′-3′)
gagugagcgacugugcccaugccagaccuaacuguaaagccacagauggguuaggucuggcaugggcacaggugccuacu

SEQ ID NO: 131 - RNA, 80 nt, Artificial
  hmTau12i Full length miRNA Sequence (5′-3′)
gagugagcgaugcccaugccagaccuaaagacuguaaagccacagaugggucuuuaggucuggcaugggcacugccuacu

SEQ ID NO: 132 - RNA, 80 nt, Artificial
  hmTau13i Full length miRNA Sequence (5′-3′)
gagugagcgcgcccaugccagaccuaaagaacuguaaagccacagauggguucuuuaggucuggcaugggcaugccuacu

SEQ ID NO: 133 - RNA, 80 nt, Artificial
  hmTau14i Full length miRNA Sequence (5′-3′)
gagugagcgacccaugccagaccuaaagaaucuguaaagccacagaugggauucuuuaggucuggcaugggcugccuacu

SEQ ID NO: 134 - RNA, 80 nt, Artificial
  hmTau15i Full length miRNA Sequence (5′-3′)
gagugagcgaccaugccagaccuaaagaaugcuguaaagccacagaugggcauucuuuaggucuggcaugggugccuacu

SEQ ID NO: 135 - RNA, 80 nt, Artificial
  hmTau16i Full length miRNA Sequence (5′-3′)
gagugagcgagcaaggugcaaauagucuacacuguaaagccacagauggguguagacuauuugcaccuugccugccuacu

SEQ ID NO: 136 - RNA, 80 nt, Artificial
  hmTau17i Full length miRNA Sequence (5′-3′)
gagugagcgaaccuccaaguguggcucguuacuguaaagccacagauggguaacgagccacacuuggaggucugccuacu

SEQ ID NO: 137 - RNA, 80 nt, Artificial
  hmTau18i Full length miRNA Sequence (5′-3′)
gagugagcgagcgagaacgccaaagccaagacuguaaagccacagaugggucuuggcuuuggcguucucgcgugccuacu

SEQ ID NO: 138 - RNA, 80 nt, Artificial
  Scramble Full length miRNA Sequence (5′-3′)
gagugagcgaccgcuaagcaugcaccguucucuguaaagccacagaugggaucacuucgaacuccuggcgggugccuacu

SEQ ID NO: 139 - RNA, 22 nt, Artificial
  hTau1i Mature Guide Sequence (5′-3“)
aucacuucgaacuccuggcggg

SEQ ID NO: 140 - RNA, 22 nt, Artificial
  hTau2i Mature Guide Sequence (5′-3”)
ucuucagcuuucaggccagcgu

SEQ ID NO: 141 - RNA, 22 nt, Artificial
  hTau3i Mature Guide Sequence (5′-3“)
uugucaucgcuuccagucccgu

SEQ ID NO: 142 - RNA, 22 nt, Artificial
  hTau4i Mature Guide Sequence (5′-3”)
aucuuuuuauuuccuccgccag

SEQ ID NO: 143 - RNA, 22 nt, Artificial
  hTau5i Mature Guide Sequence (5′-3“)
uuugcuggaauccugguggcgu

SEQ ID NO: 144 - RNA, 22 nt, Artificial
  hTau6i Mature Guide Sequence (5′-3”)
uugcuuuuacugaccaugcgag

SEQ ID NO: 145 - RNA, 22 nt, Artificial
  hTau7i Mature Guide Sequence (5′-3“)
aucuuccaucacuucgaacucc

SEQ ID NO: 146 - RNA, 22 nt, Artificial
  hTau8i Mature Guide Sequence (5′-3”)
uacugaccaugcgagcuugggu

SEQ ID NO: 147 - RNA, 22 nt, Artificial
  hTau9i Mature Guide Sequence (5′-3“)
uuuaccaucagcccccuuggcu

SEQ ID NO: 148 - RNA, 22 nt, Artificial
  hTau10i Mature Guide Sequence (5′-3”)
ucuaccaugucgaugcugccgg

SEQ ID NO: 149 - RNA, 22 nt, Artificial
  hTau11i Mature Guide Sequence (5′-3“)
uuguacacgaucuccgccccgu

SEQ ID NO: 150 - RNA, 22 nt, Artificial
  mTau1i Mature Guide Sequence (5′-3”)
uugugucaaacuccuggcgagg

SEQ ID NO: 151 - RNA, 22 nt, Artificial
  mTau2i Mature Guide Sequence (5′-3“)
auugugucaaacuccuggcgag

SEQ ID NO: 152 - RNA, 22 nt, Artificial
  mTau3i Mature Guide Sequence (5′-3”)
cauugugucaaacuccuggcga

SEQ ID NO: 153 - RNA, 22 nt, Artificial
  mTau4i Mature Guide Sequence (5′-3“)
uuaagccaugguccaugucucc

SEQ ID NO: 154 - RNA, 22 nt, Artificial
  mTau5i Mature Guide Sequence (5′-3”)
uugagucacaugcccagcggcu

SEQ ID NO: 155 - RNA, 22 nt, Artificial
  mTau6i Mature Guide Sequence (5′-3“)
uuuccuguccugucuuugcugg

SEQ ID NO: 156 - RNA, 22 nt, Artificial
  mTau7i Mature Guide Sequence (5′-3”)
acuuauacacaauuucugcucc

SEQ ID NO: 157 - RNA, 22 nt, Artificial
  mTau8i Mature Guide Sequence (5′-3“)
uuuggcauucucccugaagguc

SEQ ID NO: 158 - RNA, 22 nt, Artificial
  mTau9i Mature Guide Sequence (5′-3”)
auuuccuguccugucuuugcug

SEQ ID NO: 159 - RNA, 22 nt, Artificial
  mTau10i Mature Guide Sequence (5′-3“)
uuuggugguucaccugacccug

SEQ ID NO: 160 - RNA, 22 nt, Artificial
  mTau11i Mature Guide Sequence (5′-3”)
uucuucucgucauuuccugucc

SEQ ID NO: 161 - RNA, 22 nt, Artificial
  mTau12i Mature Guide Sequence (5′-3“)
uuuuggugguucaccugacccu

SEQ ID NO: 162 - RNA, 22 nt, Artificial
  mTau13i Mature Guide Sequence (5′-3”)
auuuuggugguucaccugaccc

SEQ ID NO: 163 - RNA, 22 nt, Artificial
  mTau14i Mature Guide Sequence (5′-3“)
gauuuuggugguucaccugacc

SEQ ID NO: 164 - RNA, 22 nt, Artificial
  mTau15i Mature Guide Sequence (5′-3”)
uguagccgcuucguucuccgga

SEQ ID NO: 165 - RNA, 22 nt, Artificial
  mTau16i Mature Guide Sequence (5′-3“)
acuagcugauggugacuuaggg

SEQ ID NO: 166 - RNA, 22 nt, Artificial
  mTau17i Mature Guide Sequence (5′-3”)
aguagagccaaucuucgaccug

SEQ ID NO: 167 - RNA, 22 nt, artificial
  mTau18i Mature Guide Sequence (5′-3“)
uuugcaccuugccaccuccugg

SEQ ID NO: 168 - RNA, 22 nt, Artificial
  mTau19i Mature Guide Sequence (5′-3”)
auuugcaccuugccaccuccug

SEQ ID NO: 169 - RNA, 22 nt, Artificial
  hmTau1i Mature Guide Sequence (5′-3“)
acauugcugaggugccguggag

SEQ ID NO: 170 - RNA, 22 nt, Artificial
  hmTau2i Mature Guide Sequence (5′-3”)
uuucaaucuucuuauucccucc

SEQ ID NO: 171 - RNA, 22 nt, Artificial
  hmTau3i Mature Guide Sequence (5′-3“)
aaucuucgacuggacucugucc

SEQ ID NO: 172 - RNA, 22 nt, Artificial
  hmTau4i Mature Guide Sequence (5′-3”)
gauuuuacuuccaccuggccac

SEQ ID NO: 173 - RNA, 22 nt, Artificial
  hmTau5i Mature Guide Sequence (5′-3“)
auuuuacuuccaccuggccacc

SEQ ID NO: 174 - RNA, 22 nt, Artificial
  hmTau6i Mature Guide Sequence (5′-3”)
ugauuuuacuuccaccuggcca

SEQ ID NO: 175 - RNA, 22 nt, Artificial
  hmTau7i Mature Guide Sequence (5′-3“)
uuacuuccaccuggccaccucc

SEQ ID NO: 176 - RNA, 22 nt, Artificial
  hmTau8i Mature Guide Sequence (5′-3”)
ugauggauguucccuaacgagc

SEQ ID NO: 177 - RNA, 22 nt, Artificial
  hmTau9i Mature Guide Sequence (5′-3“)
acauugcugagaugccguggag

SEQ ID NO: 178 - RNA, 22 nt, Artificial
  hmTau10i Mature Guide Sequence (5′-3”)
ugauggauguugccuaaugagc

SEQ ID NO: 179 - RNA, 22 nt, Artificial
  hmTau11i Mature Guide Sequence (5′-3“)
uuaggucuggcaugggcacagg

SEQ ID NO: 180 - RNA, 22 nt, Artificial
  hmTau12i Mature Guide Sequence (5′-3”)
ucuuuaggucuggcaugggcac

SEQ ID NO: 181 - RNA, 22 nt, Artificial
  hmTau13i Mature Guide Sequence (5′-3“)
uucuuuaggucuggcaugggca

SEQ ID NO: 182 - RNA, 22 nt, Artificial
  hmTau14i Mature Guide Sequence (5′-3”)
auucuuuaggucuggcaugggc

SEQ ID NO: 183 - RNA, 22 nt, Artificial
  hmTau15i Mature Guide Sequence (5′-3“)
cauucuuuaggucuggcauggg

SEQ ID NO: 184 - RNA, 22 nt, Artificial
  hmTau16i Mature Guide Sequence (5′-3”)
uguagacuauuugcaccuugcc

SEQ ID NO: 185 - RNA, 22 nt, Artificial
  hmTau17i Mature Guide Sequence (5′-3“)
uaacgagccacacuuggagguc

SEQ ID NO: 186 - RNA, 22 nt, Artificial
  hmTau18i Mature Guide Sequence (5′-3”)
ucuuggcuuuggcguucucgcg

SEQ ID NO: 187 - RNA, 22 nt, Artificial
  Scramble Mature Guide Sequence (5′-3“)
aucacuucgaacuccuggcggg

SEQ ID NO: 188 - DNA, 19 bp, Artificial
  Loop Sequence
ctgtaaagccacagatggg

SEQ ID NO: 189 - RNA, 19 nt, Artificial
  Loop Sequence
cuguaaagccacagauggg

SEQ ID NO: 190 - DNA, 9 bp, Artificial
  5′ overhang sequence
gagtgagcg

SEQ ID NO: 191 - RNA, 9 nt, Artificial
  5′ overhang sequence
gagugagcg

SEQ ID NO: 192 - DNA, 8 bp, Artificial
  3′ overhang sequence
tgcctact

SEQ ID NO: 193 - RNA, 8 nt, Artificial
  3′ overhang sequence
ugccuacu